SEQUENCE LISTING (43 sequences)

SEQ ID NO: 1 - protein, 7 aa, Artificial Sequence
  Synthetic construct
GVLPGVG

SEQ ID NO: 2 - protein, 6 aa, Artificial Sequence
  Synthetic construct
GAGVPG

SEQ ID NO: 3 - protein, 9 aa, Artificial Sequence
  Synthetic construct
VPGFGAGAG

SEQ ID NO: 4 - protein, 9 aa, Artificial Sequence
  Synthetic construct
VPGLGAGAG

SEQ ID NO: 5 - protein, 9 aa, Artificial Sequence
  Synthetic construct
VPGLGVGAG

SEQ ID NO: 6 - protein, 8 aa, Artificial Sequence
  Synthetic construct
GVLPGVGG

SEQ ID NO: 7 - protein, 5 aa, Artificial Sequence
  Synthetic construct
GVLPG

SEQ ID NO: 8 - protein, 6 aa, Artificial Sequence
  Synthetic construct
GLVPGG

SEQ ID NO: 9 - protein, 5 aa, Artificial Sequence
  Synthetic construct
GLVPG

SEQ ID NO: 10 - protein, 5 aa, Artificial Sequence
  Synthetic construct
GVPLG

SEQ ID NO: 11 - protein, 6 aa, Artificial Sequence
  Synthetic construct
GIPGVG

SEQ ID NO: 12 - protein, 6 aa, Artificial Sequence
  Synthetic construct
GGVLPG

SEQ ID NO: 13 - protein, 196 aa, Artificial Sequence
  Synthetic construct
GVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVG

SEQ ID NO: 14 - protein, 7 aa, Artificial Sequence
  Synthetic construct
GVGVLPG

SEQ ID NO: 15 - protein, 4 aa, Artificial Sequence
  Synthetic construct
GVPG

SEQ ID NO: 16 - protein, 392 aa, Artificial Sequence
  Synthetic construct
GVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVG

SEQ ID NO: 17 - protein, 784 aa, Artificial Sequence
  Synthetic construct
GVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVG

SEQ ID NO: 18 - protein, 56 aa, Artificial Sequence
  Synthetic construct
TTYKLVINGKTLKGETTTKAVDAETAEKAFKQYANDNGVDGVWTYDDATKTFTVTE

SEQ ID NO: 19 - protein, 56 aa, Artificial Sequence
  Synthetic construct
TTYKLILNGKTLKGETTTEAVDAATAEKVFKQYANDNGVDGEWTYDDATKTFTVTE

SEQ ID NO: 20 - protein, 56 aa, Artificial Sequence
  Synthetic construct
TTYKLVINGKTLKGETTTEAVDAATAEKVFKQYANDNGVDGEWTYDDATKTFTVTE

SEQ ID NO: 21 - protein, 56 aa, Artificial Sequence
  Synthetic construct
TTYKLVINGKTLKGETTTKAVDAETAAAAFAQYANDNGVDGVWTYDDATKTFTVTE

SEQ ID NO: 22 - protein, 56 aa, Artificial Sequence
  Synthetic construct
TTYKLVINGKTLKGETTTKAVDAETAAAAFAQYARRNGVDGVWTYDDATKTFTVTE

SEQ ID NO: 23 - protein, 199 aa, Artificial Sequence
  Synthetic construct
GGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGG

SEQ ID NO: 24 - protein, 423 aa, Artificial Sequence
  Synthetic construct
GGAGVPGGAGVPGGAGVPGGAGVPGGAGVPGGAGVPGGAGVPGGAGVPGGAGVPGGAGVPGGAGVPGGAGVPGGAGVPGGAGVPGGAGVPGGAGVPGGAGVPGGAGVPGGAGVPGGAGVPGGAGVPGGAGVPGGAGVPGGAGVPGGAGVPGGAGVPGGAGVPGGAGVPGGAGVPGGAGVPGGAGVPGGAGVPGGAGVPGGAGVPGGAGVPGGAGVPGGAGVPGGAGVPGGAGVPGGAGVPGGAGVPGGAGVPGGAGVPGGAGVPGGAGVPGGAGVPGGAGVPGGAGVPGGAGVPGGAGVPGGAGVPGGAGVPGGAGVPGGAGVPGGAGVPGGAGVPGGAGVPGGAGVPGGAGVPGGAGVPGGAGVPGGAGVPGGAGVPGGAGVPGGAGVPGGAGVPGGAGVPGGAGVPGGAGVPGGAGVPGGG

SEQ ID NO: 25 - protein, 192 aa, Artificial Sequence
  Synthetic construct
GVPGFGAGAGVPGFGAGAGVPGFGAGAGVPGFGAGAGVPGFGAGAGVPGFGAGAGVPGFGAGAGVPGFGAGAGVPGFGAGAGVPGFGAGAGVPGFGAGAGVPGFGAGAGVPGFGAGAGVPGFGAGAGVPGFGAGAGVPGFGAGAGVPGFGAGAGVPGFGAGAGVPGFGAGAGVPGFGAGAGVPGFGAGAGGG

SEQ ID NO: 26 - protein, 867 aa, Artificial Sequence
  Synthetic construct
GVPGLGAGAGVPGLGAGAGVPGLGAGAGVPGLGAGAGVPGLGAGAGVPGLGAGAGVPGLGAGAGVPGLGAGAGVPGLGAGAGVPGLGAGAGVPGLGAGAGVPGLGAGAGVPGLGAGAGVPGLGAGAGVPGLGAGAGVPGLGAGAGVPGLGAGAGVPGLGAGAGVPGLGAGAGVPGLGAGAGVPGLGAGAGVPGLGAGAGVPGLGAGAGVPGLGAGAGVPGLGAGAGVPGLGAGAGVPGLGAGAGVPGLGAGAGVPGLGAGAGVPGLGAGAGVPGLGAGAGVPGLGAGAGVPGLGAGAGVPGLGAGAGVPGLGAGAGVPGLGAGAGVPGLGAGAGVPGLGAGAGVPGLGAGAGVPGLGAGAGVPGLGAGAGVPGLGAGAGVPGLGAGAGVPGLGAGAGVPGLGAGAGVPGLGAGAGVPGLGAGAGVPGLGAGAGVPGLGAGAGVPGLGAGAGVPGLGAGAGVPGLGAGAGVPGLGAGAGVPGLGAGAGVPGLGAGAGVPGLGAGAGVPGLGAGAGVPGLGAGAGVPGLGAGAGVPGLGAGAGVPGLGAGAGVPGLGAGAGVPGLGAGAGVPGLGAGAGVPGLGAGAGVPGLGAGAGVPGLGAGAGVPGLGAGAGVPGLGAGAGVPGLGAGAGVPGLGAGAGVPGLGAGAGVPGLGAGAGVPGLGAGAGVPGLGAGAGVPGLGAGAGVPGLGAGAGVPGLGAGAGVPGLGAGAGVPGLGAGAGVPGLGAGAGVPGLGAGAGVPGLGAGAGVPGLGAGAGVPGLGAGAGVPGLGAGAGVPGLGAGAGVPGLGAGAGVPGLGAGAGVPGLGAGAGVPGLGAGAGVPGLGAGAGVPGLGAGAGVPGLGAGAGVPGLGAGAGVPGLGAGAGGG

SEQ ID NO: 27 - protein, 9 aa, Artificial Sequence
  Synthetic construct
  MISC_FEATURE (1)..(1): one or more glycine amino acid residues
  MISC_FEATURE (9)..(9): one or more glycine amino acid residues
VPGLGVGAG

SEQ ID NO: 28 - protein, 5 aa, Artificial Sequence
  Synthetic construct
GGVPG

SEQ ID NO: 29 - protein, 56 aa, Artificial Sequence
  Synthetic construct
TTYKLVIAGKTLKGETTTEAVDAATAEKVFKQYANDAGVDGEWTYDDATKTFTVTE

SEQ ID NO: 30 - protein, 40 aa, Artificial Sequence
  Synthetic construct
TTEAVDAATAEKVFKQYANDNGVDGEWTYDDATKTFTVTE

SEQ ID NO: 31 - protein, 25 aa, Artificial Sequence
  Synthetic construct
QYANDNGVDGEWTYDDATKTFTVTE

SEQ ID NO: 32 - protein, 19 aa, Artificial Sequence
  Synthetic construct
EKVFKQYANDNGVDGEWTY

SEQ ID NO: 33 - protein, 11 aa, Artificial Sequence
  Synthetic construct
NDNGVDGEWTY

SEQ ID NO: 34 - protein, 5 aa, Artificial Sequence
  Synthetic construct
GGGGS

SEQ ID NO: 35 - protein, 5 aa, Artificial Sequence
  Synthetic construct
GGGGC

SEQ ID NO: 36 - protein, 257 aa, Artificial Sequence
  Synthetic construct
GVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGGGGSTTYKLVINGKTLKGETTTKAVDAETAEKAFKQYANDNGVDGVWTYDDATKTFTVTE

SEQ ID NO: 37 - protein, 460 aa, Artificial Sequence
  Synthetic construct
GVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGGGGSTTYKLVINGKTLKGETTTKAVDAETAEKAFKQYANDNGVDGVWTYDDATKTFTVTE

SEQ ID NO: 38 - protein, 845 aa, Artificial Sequence
  Synthetic construct
GVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGGGGSTTYKLVINGKTLKGETTTKAVDAETAEKAFKQYANDNGVDGVWTYDDATKTFTVTE

SEQ ID NO: 39 - protein, 20 aa, Artificial Sequence
  Synthetic construct
GGGGCGGGGCGGGGCGGGGC

SEQ ID NO: 40 - protein, 20 aa, Artificial Sequence
  Synthetic construct
GGGGCGGGGCGGGGCGGGGC

SEQ ID NO: 41 - protein, 277 aa, Artificial Sequence
  Synthetic construct
GVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGGGGCGGGGCGGGGCGGGGCGGGGSTTYKLVINGKTLKGETTTKAVDAETAEKAFKQYANDNGVDGVWTYDDATKTFTVTE

SEQ ID NO: 42 - protein, 473 aa, Artificial Sequence
  Synthetic construct
GVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGGGGCGGGGCGGGGCGGGGCGGGGSTTYKLVINGKTLKGETTTKAVDAETAEKAFKQYANDNGVDGVWTYDDATKTFTVTE

SEQ ID NO: 43 - protein, 865 aa, Artificial Sequence
  Synthetic construct
GVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGVLPGVGGGGGCGGGGCGGGGCGGGGCGGGGSTTYKLVINGKTLKGETTTKAVDAETAEKAFKQYANDNGVDGVWTYDDATKTFTVTE

(19) United States
(12) Patent Application Publication
(10) Pub. No.: US 2022/0280615 A1
(43) Pub. Date: Sep. 8, 2022

(54) IMMUNE TOLERANT ELASTIN-LIKE RECOMBINANT PEPTIDES AND METHODS OF USE

(71) Applicant: University of Utah Research Foundation, Salt Lake City, UT (US)

(72) Inventors: Mingnan Chen, Salt Lake City, UT (US); Peng Wang, Salt Lake City, UT (US); Peng Zhao, Salt Lake City, UT (US); Shuyun Dong, Salt Lake City, UT (US)

(21) Appl. No.: 17/637,639

(22) PCT Filed: Jun. 30, 2020
(86) PCT No.: PCT/US2020/040230

§ 371 Date: Feb. 23, 2022

Related U.S. Application Data
(60) Provisional application No. 62/890,936, filed on Aug. 23, 2019.

(51) Int. Cl. A61K 38/39; A61K 47/64; A61K 47/42; A61K 9/00; A61K 47/02; A61P 35/00

(52) U.S. Cl. CPC A61K 38/39; A61K 47/64; A61K 47/42; A61K 9/0019; A61K 47/02; A61P 35/00

(57) ABSTRACT

Disclosed herein, are recombinant polypeptides comprising one or more homologous amino acid repeats fused with an IgG binding domain The recombinant polypeptides can be bound to a therapeutic antibody and used a delivery vehicle to increase the retention time and reduce systemic-related side effects of the therapeutic antibodies. Also disclosed herein are pharmaceutical compositions including the recombinant polypeptides bound to a therapeutic antibody; and methods of administering the same to patients

### CROSS REFERENCE TO RELATED APPLICATIONS

This application claims the benefit of the filing date of U.S. Provisional Application 62/890,936, which was filed on Aug. 23, 2019. The content of this earlier filed application is hereby incorporated by reference herein in its entirety.

### INCORPORATION OF THE SEQUENCE LISTING

The present application contains a sequence listing that was submitted in ASCII format via EFS-Web concurrent with the filing of the application, containing the file name 21101_0378P1_Sequence_Listing which is 65,536 bytes in size, created on May 29, 2020, and is herein incorporated by reference in its entirety.

### BACKGROUND

Immune checkpoint antibodies can be used to treat a variety of cancers. To date, the clinical immune checkpoint antibodies available are intravenously administered. Systemic administration of immune checkpoint antibodies is effective in controlling the disseminated tumor. However, when the tumor is confined to a local area, systemic antibody treatment is not efficient and often associated with side effects. In such cases, local delivery of immune checkpoint antibodies may provide benefits by increasing the treatment efficacy and reducing the side effects. Without a delivery system, however, the locally administered antibodies are subject to short retention time at local areas and high exposure to the systemic circulation. These challenges make local immune checkpoint antibody treatment less promising as expected. Thus, alternative methods to deliver immune checkpoint antibodies locally is needed.

### SUMMARY

Disclosed herein are recombinant polypeptides comprising an homologous amino acid repeat sequence, having at least 75% amino acid sequence identity to the homologous amino acid repeat sequence, and wherein the homologous amino acid repeat sequence is: Gly-Val-Leu-Pro-Gly-Val-Gly (SEQ ID NO: 1); Gly-Ala-Gly-Val-Pro-Gly (SEQ ID NO: 2); Val-Pro-Gly-Phe-Gly-Ala-Gly-Ala-Gly (SEQ ID NO: 3); Val-Pro-Gly-Leu-Gly-Ala-Gly-Ala-Gly (SEQ ID NO: 4); Val-Pro-Gly-Leu-Gly-Val-Gly-Ala-Gly (SEQ ID NO: 5); Gly-Val-Leu-Pro-Gly-Val-Gly-Gly (SEQ ID NO: 6); Gly-Val-Leu-Pro-Gly (SEQ ID NO: 7); Gly-Leu-Val-Pro-Gly-Gly (SEQ ID NO: 8); Gly-Leu-Val-Pro-Gly (SEQ ID NO: 9); Gly-Val-Pro-Leu-Gly (SEQ ID NO: 10); Gly-Ile-Pro-Gly-Val-Gly (SEQ ID NO: 11); Gly-Gly-Val-Leu-Pro-Gly (SEQ ID NO: 12); Gly-Val-Gly-Val-Leu-Pro-Gly (SEQ ID NO: 14); or Gly-Val-Pro-Gly (SEQ ID NO: 15); and an IgG binding domain.

Disclosed herein are methods of increasing the efficacy of a therapeutic agent or increasing the half of a therapeutic agent in a subject, the methods comprising administering to the subject a therapeutic agent conjugated to a recombinant polypeptide, wherein the recombinant polypeptide comprises an homologous amino acid repeat sequence covalently linked to a IgG binding domain, and wherein the therapeutic agent is non-covalently conjugated to the IgG binding domain, and wherein the conjugate is administered by direct injection, whereby the efficacy or half-life of the therapeutic agent is increased.

### BRIEF DESCRIPTION OF THE DRAWINGS

FIGS. 1A-E show the characterization of the Tt of the iTEP-IBD polypeptide and the binding between the iTEP-IBD polypeptide and antibodies. FIG. 1A is a reprehensive plot showed the turbidity of the iTEP-IBD polypeptide solution over the change of temperature. The turbidity of the solution was characterized by the absorbance at 350 nm. FIG. 1B shows the Tt of each iTEP-IBD polypeptide was dependent on its concentration (n=3 biologically independent samples, one-way ANOVA with Tukey post hoc test). FIG. 1C shows the iTEP-IBD polypeptide bound to IgG and trapped IgG in depots. The percentage of IgG in depots was dependent on the ratio of the iTEP-IBD polypeptide to IgG (n=5 biologically independent samples, one-way ANOVA with Tukey post hoc test). FIG. 1D shows the iTEP-IBD polypeptide did not impact the target-binding ability of the αPD-1 antibody. Free αPD-1 antibody and the iTEP₁₁₂-IBD/αPD-1 polypeptide stained target cells similarly (n=6 biologically independent samples, unpaired two-tailed t-test). FIG. 1E is a representative flow cytometry plot showed the comparable target-binding abilities of the αPD-1 antibody and the iTEP₁₁₂-IBD/αPD-1 polypeptide. Data were shown as mean±standard deviation (SD). ****P<0.0001, NS=not significant.

FIGS. 2A-D show the release profile and low plasma concentration of the iTEP₁₁₂-IBD/IgG. FIG. 2A shows the in vitro release curves of IgG from the iTEP₁₁₂-IBD/IgG depots in PBS or mouse serum (n=3 biologically independent samples, unpaired two-tailed t-test). FIG. 2B shows the fluorescent IVIS imaging of mice that were subcutaneously injected with IgG or the iTEP₁₁₂-IBD/IgG (n=5 mice). The ratio of the iTEP₁₁₂-IBD polypeptide to IgG was 8:1. The presence of labeled IgG was indicated by the yellow/red color on the image. FIG. 2C shows the quantification of the radiant efficiency of remaining IgG in mice as shown in FIG. 2B (n=5 mice, unpaired two-tailed t-test). The radiant efficiency at each time point was normalized to the initial radiant efficiency. The release half-life (t_{1/2}) was calculated by fitting the time and the normalized radiant efficiency to the first-order release model. FIG. 2D shows mouse plasma concentration of sulfo-cyanine7-labeled IgG over time when the IgG was injected solely or together with the iTEP₁₁₂-IBD=5 mice, unpaired two-tailed t-test). The ratio of the iTEP₁₁₂-IBD polypeptide to IgG was 8:1. Data were shown as mean±SD. *P<0.5, ****P<0.0001.

FIGS. 3A-B show the in vivo release profile of the iTEP₂₈-IBD/IgG and the iTEP₅₆-IBD/IgG. FIG. 3A shows fluorescent IVIS imaging of mice injected with the iTEP₂₈-IBD/IgG or the iTEP₅₆-IBD/IgG (n=5 mice). The ratio of the iTEP₂₈-IBD polypeptide and the iTEP₅₆-IBD polypeptide to IgG was 8:1. FIG. 3B shows the quantification of radiant efficiency of remaining IgG at injection sites as shown in FIG. 3A (n=5 mice, unpaired two-tailed t-test). Data were shown as mean±SD. **P<0.01.

FIGS. 4A-D shows the characterization of the iTEP-C-IBD polypeptide and the in vivo release profile of the iTEP-C-IBD/IgG. FIG. 4A is a reprehensive plot showed the turbidity of the iTEP-C-IBD polypeptide solution versus temperature. FIG. 4B is a plot showing the concentration dependence of Tt of the iTEP-C-IBD polypeptide (n=3 biologically independent samples, one-way ANOVA with Tukey post hoc test). FIG. 4C shows the iTEP-C-IBD polypeptide trapped IgG in depots (n=5 biologically independent samples, one-way ANOVA with Tukey post hoc test). FIG. 4D shows fluorescent IVIS imaging of mice injected with the iTEP₂₈-C-IBD/IgG, the iTEP₅₆-C-IBD/IgG, or the iTEP₁₁₂-C-IBD/IgG (n=5 mice). The ratio of the iTEP-C-IBD polypeptide to IgG was 8:1. FIG. 4E shows the quantification of radiant efficiency of remaining IgG over time as shown in FIG. 4D (n=5 mice, one-way ANOVA with Tukey post hoc test). Data were shown as mean±SD. **P<0.01, ****P<0.0001, NS=not significant.

FIGS. 5A-B shows the in vivo release profile of the iTEP-C-IBD/IgG at the ratio of 32:1. FIG. 5A shows fluorescent IVIS imaging of mice injected with the iTEP₂₈-C-IBD/IgG, the iTEP₅₆-C-IBD/IgG, or the iTEP₁₁₂-C-IBD/IgG (n=5 mice). The ratio of the iTEP-C-IBD polypeptide to IgG was 32:1. FIG. 5B shows the quantification of radiant efficiency of remaining IgG at injection sites as shown in FIG. 5A (n=5 mice, one-way ANOVA with Tukey post hoc test). Data were shown as mean±SD. ***P<0.001, NS=not significant.

FIGS. 6A-E shows the distribution of the iTEP₁₁₂-C-IBD/IgG in blood, tumor, and other organs. FIG. 6A shows the fluorescent IVIS imaging of tumors that were injected with free IgG or the iTEP₁₁₂-C-IBD/IgG at 24 and 72 hours after injection (n=5 mice). The ratio of the iTEP₁₁₂-C-IBD polypeptide to IgG was 8:1. FIG. 6B shows the accumulation of IgG in tumors that were directly injected with free IgG or the iTEP₁₁₂-C-IBD/IgG (n=5 mice, unpaired two-tailed t-test). The data were expressed as the percentage of injected dose per gram of tissue, (ID%)/gram. The accumulation of IgG at spleen, liver, kidney, and lung at 24 hours (FIG. 6C) and 72 hours (FIG. 6D) after injection (n=5 mice, unpaired two-tailed t-test). FIG. 6E shows the mouse serum concentration of injected IgG at 24 and 72 hours after injection (n=5 mice, unpaired two-tailed t-test). Data were shown as mean±SD. *P<0.5, **P<0.01, ***P<0.001, ****P<0.0001.

FIGS. 7A-E shows the in vivo release kinetics of IgG and the iTEP₁₁₂-IBD/IgG using different mathematical models. Data collected at each time point was normalized to the data collected immediately after the injection when it was considered as time zero. Zero-order model (FIG. 7A), first-order model (FIG. 7B), Higuchi model (FIG. 7C), Hixson-Crowell model (FIG. 7D), and Korsmeyer-Peppas model (FIG. 7E) were used to analyze the release kinetics. Equation and coefficient of determination (R²) of each fitted line were displayed on each plot.

FIGS. 8A-B show the standard curves of labeled IgG. The curves showed the linear correlation between the fluorescent intensity and the concentration of the fluorescein-labeled IgG (FIG. 8A) and sulfo-cyanine7-labeled IgG (FIG. 8B) in PBS solution. Equation and coefficient of determination (R²) of each line were displayed on the plots. The concentrations of IgG in both standard curves from low to high were 0.0003, 0.0009, 0.0027, 0.0081, and 0.0243 mg/mL. The fluorescent signal of the lowest IgG concentration in the standard curves was 20-fold (FIG. 8A) and 6-fold (FIG. 8B) higher than the background signal. The fluorescent background of plasma, serum, and other tissues was subtracted before the standard curves were used to calculate the IgG concentration in these biological components.

### DETAILED DESCRIPTION

The present disclosure can be understood more readily by reference to the following detailed description of the invention, the figures and the examples included herein.

Before the present methods and compositions are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

Moreover, it is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including matters of logic with respect to arrangement of steps or operational flow, plain meaning derived from grammatical organization or punctuation, and the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

Definitions

As used in the specification and the appended claims, the singular forms “a,” “an” and “the” include plural referents unless the context clearly dictates otherwise.

The word “or” as used herein means any one member of a particular list and also includes any combination of members of that list.

Ranges can be expressed herein as from “about” or “approximately” one particular value, and/or to “about” or “approximately” another particular value. When such a range is expressed, a further aspect includes from the one particular value and/or to the other particular value. Similarly, when values are expressed as approximations, by use of the antecedent “about,” or “approximately,” it will be understood that the particular value forms a further aspect. It will be further understood that the endpoints of each of the ranges are significant both in relation to the other endpoint and independently of the other endpoint. It is also understood that there are a number of values disclosed herein and that each value is also herein disclosed as “about” that particular value in addition to the value itself. For example, if the value “10” is disclosed, then “about 10” is also disclosed. It is also understood that each unit between two particular units is also disclosed. For example, if 10 and 15 are disclosed, then 11, 12, 13, and 14 are also disclosed.

As used herein, the terms “optional” or “optionally” mean that the subsequently described event or circumstance may or may not occur and that the description includes instances where said event or circumstance occurs and instances where it does not.

As used herein, the term “sample” is meant a tissue or organ from a subject; a cell (either within a subject, taken directly from a subject, or a cell maintained in culture or from a cultured cell line); a cell lysate (or lysate fraction) or cell extract; or a solution containing one or more molecules derived from a cell or cellular material (e.g. a polypeptide or nucleic acid), which is assayed as described herein. A sample may also be any body fluid or excretion (for example, but not limited to, blood, urine, stool, saliva, tears, bile) that contains cells or cell components.

As used herein, the term “subject” refers to the target of administration, e.g., a human. Thus the subject of the disclosed methods can be a vertebrate, such as a mammal, a fish, a bird, a reptile, or an amphibian. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). In one aspect, a subject is a mammal. In another aspect, a subject is a human. The term does not denote a particular age or sex. Thus, adult, child, adolescent and newborn subjects, as well as fetuses, whether male or female, are intended to be covered.

As used herein, the term “patient” refers to a subject afflicted with a disease or disorder. The term “patient” includes human and veterinary subjects. In some aspects of the disclosed methods, the “patient” has been diagnosed with cancer. In some aspects of the disclosed methods, the “patient” has been identified as being in need for treatment for cancer, such as, for example, prior to administering a therapeutic agent to the patient.

As used herein, the term “comprising” can include the aspects “consisting of” and “consisting essentially of.”

As used herein the terms “amino acid” and “amino acid identity” refers to one of the 20 naturally occurring amino acids or any non-natural analogues that may be in any of the variants, peptides or fragments thereof disclosed. Thus “amino acid” as used herein means both naturally occurring and synthetic amino acids. For example, homophenylalanine, citrulline and norleucine are considered amino acids for the purposes of the invention. “Amino acid” also includes amino acid residues such as proline and hydroxyproline. The side chain may be in either the (R) or the (S) configuration. If non-naturally occurring side chains are used, non-amino acid substituents may be used, for example to prevent or retard in vivo degradation.

The term “fragment” can refer to a portion (e.g., at least 4, 5, 6, 7, 8, 9, 10, 11, 12, 13, 14, 15, 16, 17, 18, 19, 20, 21, 22, 23, 24, 25, 26, 27, 28, 29, 30, etc. amino acids) of a peptide that is substantially identical to a reference peptide and retains the biological activity of the reference. In some aspects, the fragment or portion retains at least 50%, 75%, 80%, 85%, 90%, 95% or 99% of the biological activity of the reference peptide described herein. Further, a fragment of a referenced peptide can be a continuous or contiguous portion of the referenced polypeptide (e.g., a fragment of a peptide that is ten amino acids long can be any 2-9 contiguous residues within that peptide).

A “variant” can mean a difference in some way from the reference sequence other than just a simple deletion of an N- and/or C-terminal amino acid residue or residues. Where the variant includes a substitution of an amino acid residue, the substitution can be considered conservative or non-conservative. Conservative substitutions are those within the following groups: Ser, Thr, and Cys; Leu, Ile, and Val; Glu and Asp; Lys and Arg; Phe, Tyr, and Trp; and Gln, Asn, Glu, Asp, and His. Variants can include at least one substitution and/or at least one addition, there may also be at least one deletion. Variants can also include one or more non-naturally occurring residues. For example, variants may include selenocysteine (e.g., seleno-L-cysteine) at any position, including in the place of cysteine. Many other “unnatural” amino acid substitutes are known in the art and are available from commercial sources. Examples of non-naturally occurring amino acids include D-amino acids, amino acid residues having an acetylaminomethyl group attached to a sulfur atom of a cysteine, a pegylated amino acid, and omega amino acids of the formula NH2(CH2)ₙCOOH wherein n is 2-6 neutral, nonpolar amino acids, such as sarcosine, t-butyl alanine, t-butyl glycine, N-methyl isoleucine, and norleucine. Phenylglycine may substitute for Trp, Tyr, or Phe; citrulline and methionine sulfoxide are neutral nonpolar, cysteic acid is acidic, and ornithine is basic. Proline may be substituted with hydroxyproline and retain the conformation conferring properties of proline.

As used herein, the term “iTEP” refers to an immune-tolerant, elastin-like polypeptide. iTEPs can differ from previously disclosed elastin-like polypeptides (referred to as ELPs; ELPs are described in D. M. Floss, et al., Elastin-like polypeptides revolutionize recombinant protein expression and their biomedical application, Trends Biotechnol. 28(1) (2010) 37-45; and T. Kowalczyk, et al., Elastin-like polypeptides as a promising family of genetically-engineered protein based polymers, World J. Microbiol. Biotechnol. 30(8) (2014) 2141-52.). iTEPs have a phase transition property and are immune-tolerant. The iTEP sequences disclosed herein can be referred to as a homologous amino acid sequence that can be repeated, for example, 20 to 120 times, and fused to an IgG binding domain to form one or more of the recombinant polypeptides disclosed herein. In some aspects, the iTEP sequence can be fused to an IgG binding domain (e.g., IBD) via a linker. In some aspects, the term “iTEP-IBD polypeptide” encompasses a linker sequence between the iTEP sequence and the IBD.

Introduction

Monoclonal antibodies (e.g., IgGs) are widely used in medicine. It is often desired to limit the distribution of therapeutic IgGs inside target tissues because this increases bioavailability of the IgGs to target cells while reducing the exposure of the therapeutic IgGs to other tissues and cells. The exposure of the IgGs to other tissues and cells often results in side effects. To increase the distribution and the accumulation of the IgGs inside target tissues, the IgGs have been directly injected into the tissues. However, the injected IgGs quickly diffuse outside of the tissues. Disclosed herein are compositions and methods for increasing the retention time and retention amount of IgGs in tissues. The recombinant polypeptides and compositions disclosed herein can comprise immune-tolerant elastin-like peptides (iTEPs) and an IgG binding domain. In some aspects, the recombinant polypeptides comprising a homologous amino acid repeat (e.g., an iTEP and an IgG binding domain which can be referred to as a “Paced IgG Emitter” or “PIE”).The recombinant polypeptides and compositions described herein can form coacervates inside the body, which can be triggered by physiological temperature. The coacervates can be used to store IgGs inside the tissues in which the coacervates form. The recombinant polypeptides, compositions and methods disclosed herein can have two elements: coacervates assembled from iTEPs (also referred to herein as homologous amino acid repeat sequences) and an IgG binding domain that can be attached with the iTEPs to form a polypeptide, a fusion polypeptide or a recombinant polypeptide that can then be used to bind an IgG. Functionally, the retention of the IgGs as bound to the fusion or recombinant polypeptides disclosed herein or the release of therapeutic IgGs from the fusion or recombinant polypeptide disclosed herein can be determined by at least but not limited to the following factors, the sequence/hydrophobicity of iTEP (or homologous amino acid repeat sequence), the ratio between the IgG and homologous amino acid repeat in the disclosed recombinant polypeptide, and the cross-linking status between homologous amino acid repeat sequences. The cross-linking status and hydrophobicity can also determine the stability of the recombinant polypeptides. A variety of recombinant polypeptides can be designed and generated by modulating these factors and are described herein.

The advantages of using the recombinant polypeptides and compositions described herein to deliver therapeutic agents (e.g., therapeutic antibodies or IgGs) as compared to other methods that increase IgG retention in target tissues include but are not limited to the following.. First, there is no need to modify the IgG (e.g., therapeutic agent or antibody) to utilize the recombinant polypeptides described herein; other methods require a modification of the IgG which adds at least one more step into the preparation procedure. In addition, modification of the IgG may compromise the function of the IgGs. Second, the fusion of iTEPs or homologous amino acid repeats and the IgG binding domain can be generated as a single recombinant protein. The fusion protein has excellent homogeneity, reproducibility, and scalability. Third, the stability of the recombinant polypeptides bound to an IgG which can determine the retention time of IgGs can be easily modulated. Thus, recombinant polypeptides bound to an IgGs can be generated such that the release kinetics of the IgGs can be controlled or diversified.

The recombinant polypeptides bound to an IgG can be used to deliver any therapeutic or diagnostic IgG that is desired to be retained in one or more specific tissues for an extended period of time. Examples of IgGs include but are not limited are cancer immune checkpoint inhibitors, such as Ipilimumab and Nivolumab. The drugs, for example, have application and efficacy in cancer treatment. However, their use has been hindered by side effects that are caused by the interaction of these drugs with immune cells that are irrelevant to cancer treatment.

Immune checkpoint antibodies represent one of the fastest growing areas of new drug development. By the end of 2018, there were seven immune checkpoint antibodies that have been approved by the U.S. Food and Drug Administration, including pembrolizumab, nivolumab, and cemiplimab that target PD-1 (R. M. Poole, Drugs 74(16) (2014) 1973-81; E. D. Deeks, Drugs 74(11) (2014) 1233-9; and A. Markham, S. Duggan, Drugs 78(17) (2018) 1841-6), atezolizumab, avelumab, and durvalumab that target PD-L1 (A. Markham, Drugs 76(12) (2016) 1227-32; E. S. Kim, Drugs 77(8) (2017) 929-37; Y. Y. Syed, Drugs 77(12) (2017) 1369-76), and ipilimumab that targets CTLA-4 (F. Cameron, et al., Drugs 71(8) (2011) 1093-104). The indications of these antibodies cover melanoma, non-small cell lung cancer (NSCLC), urothelial carcinoma, lymphoma, and so on (K. M. Hargadon, et al., Int Immunopharmacol 62 (2018) 29-39). In clinical practice, immune checkpoint antibodies are given to patients through intravenous infusion. After intravenous infusion, antibodies enter into systemic blood circulation, through which the antibodies are expected to go to the disease sites to take effect (E. D. Lobo, et al., J Pharm Sci 93(11) (2004) 2645-68). Systemic administration, such as intravenous infusion, of immune checkpoint antibodies is suitable to treat disseminated diseases, such as blood cancer (E. Jabbour, et al., Blood 125(26) (2015) 4010-6). However, when the tumor is limited to a specific area, there are challenges associated with the systemic administration of immune checkpoint antibodies. First, there are physiological barriers, such as poor blood flow, elevated interstitial fluid pressure, and the dense extracellular matrix that can restrict the access of antibodies from blood circulation to solid tumors (G. M. Thurber, et al., Adv Drug Deliv Rev 60(12) (2008) 1421-34; and M. Tabrizi, et al., AAPS J 12(1) (2010) 33-43), thus, limiting the local bioavailability of antibodies at the tumor sites (C. F. Molthoff, et al., Br J Cancer 65(5) (1992) 677-83; L. T. Baxter, et al., Cancer Res 54(6) (1994) 1517-28; and C. M. Lee, I. F. Tannock, BMC Cancer 10 (2010) 255). The tumor accumulation of intravenously injected antibodies is about 1% to 25% of the injected dose per gram of tumor in mice (C. F. Molthoff, et al., Br J Cancer 65(5) (1992) 677-83; and A. A. Epenetos, et al., Br J Cancer 46(1) (1982) 1-8). The accumulation in human patients is much lower, which is about 0.002% to 0.03% of the injected dose per gram of tumor (A. A. Epenetos, et al., Cancer Res 46(6) (1986) 3183-91; and M. R. Buist, et al., Int J Cancer 64(2) (1995) 92-8). The limited tumor bioavailability of immune checkpoint antibodies results in suboptimal therapeutic effects. A meta-analysis showed that the overall response rate of anti-PD-1 and anti-PD-L1 in patients with advanced solid tumor was 21% (A. Carretero-Gonzalez, et al., Oncotarget 9(9) (2018) 8706-15). Increasing the tumor accumulation of therapeutic antibodies would promote antitumor efficacy, as evidenced by preclinical research (A. R. Nikpoor, et al., Nanomedicine 13(8) (2017) 2671-82; and T. H. Shin, et al., Mol Cancer Ther 13(3) (2014) 651-61). Second, immune checkpoint antibodies administered systemically can go to healthy tissues through blood circulation, which may lead to undesired adverse effects (M. A. Postow, et al., N Engl J Med 378(2) (2018) 158-68; and J. M. Michot, et al., Eur J Cancer 54 (2016) 139-48). For example, 55% of melanoma patients experienced grade 3-4 side effects when they were receiving the combination therapy of anti-PD-1 and anti-CTLA-4 antibodies. The side effects were so serious that 36.4% of patients had to stop the treatment (J. Larkin, et al., N Engl J Med 373(1) (2015) 23-34). The side effects of an anti-CTLA-4 antibody appeared to be dose-dependent. The grade >3 side effects were seen in 37% of patients treating with 10 mg/kg ipilimumab and 18% of patients treating with 3 mg/kg ipilimumab (J. D. Wolchok, et al., Lancet Oncol 11(2) (2010) 155-64). By disturbing immune homeostasis in normal organs, immune checkpoint antibodies can cause organ-specific toxicity. The commonly affected organs and tissues include liver, lung, skin, gastrointestinal tract, endocrine glands and hematologic systems (A. Winer, et al., J Thorac Dis 10(Suppl 3) (2018) S480-9; and F. Martins, et al., Nat Rev Clin Oncol (2019)). Third, the systemic administration of immune checkpoint antibodies is associated with the high costs of the treatments. For example, the antibody concentrations are highly diluted after entering into blood circulation through intravenous infusion. To achieve therapeutic concentration at the disease sites, patients need to receive high doses of antibodies, which in part makes antibody treatment expensive (A.F. Shaughnessy, BMJ 345 (2012) e8346).

Given those challenges of systemic administration of antibodies, local administration of immune checkpoint antibodies may lead to some advantages for treating a localized tumor (M. F. Fransen, et al., Clin Cancer Res 19(19) (2013) 5381-9; A. Marabelle, et al., J Clin Invest 123(6) (2013) 2447-63; I. Sagiv-Barfi, et al., Sci Transl Med 10(426) (2018); V. Huynh, et al., Chembiochem 20(6) (2019) 747-53; L. C. Sandin, et al., Oncoimmunology 3(1) (2014) e27614; and L.C. Sandin, et al., Cancer Immunol Res 2(1) (2014) 80-90). For localized diseases, direct injections of antibodies to the disease sites can increase local bioavailability (R. G. Jones, A. Martino, Crit Rev Biotechnol 36(3) (2016) 506-20). High concentrations of antibodies at disease sites can be achieved, thus, increasing therapeutic effects (K. Kitamura, et al., Cancer Res 52(22) (1992) 6323-8; and A. D. Simmons, et al., Cancer Immunol Immunother 57(8) (2008) 1263-70). Due to the increased bioavailability, local administration uses much lower doses of antibodies in comparison to systemic administration. In a preclinical study, direct injection of immune checkpoint antibodies to primary tumors in mice can achieve the same or even better antitumor effects than the intravenous injection (A. Marabelle, et al., J Clin Invest 123(6) (2013) 2447-63). The doses of immune checkpoint antibodies needed for local injection was about 1% of that needed for intravenous injection. The low doses of antibodies required for local injection can reduce the high cost of antibody treatment (D. W. Grainger, Expert Opin Biol Ther 4(7) (2004) 1029-44). Since low doses of antibodies are directly injected to disease sites, the exposure of antibodies to healthy tissues will likely decrease. Therefore, local antibody injection can also decrease the risk of side effects (A. D. Simmons, et al., Cancer Immunol Immunother, 57(8) (2008) 1263-70; A. Marabelle, et al., Clin Cancer Res 19(19) (2013) 5261-3; B. Kwong, et al., Biomaterials 32(22) (2011) 5134-47; B. Kwong, S. A. et al., Cancer Res 73(5) (2013) 1547-58).

Given the results from animal studies, clinical trials have been initiated to evaluate the clinical benefits of local injection of immune checkpoint antibodies in patients. Intratumoral injection of ipilimumab and interleukin-2 was evaluated in patients with unresectable melanoma (NCT01672450). Intratumoral ipilimumab and local radiation therapy were applied in patients with recurrent melanoma, non-Hodgkin lymphoma, colon and rectal cancer (NCT01769222). A phase I/II study evaluated the intratumoral ipilimumab and toll-like receptor 9 agonist in combination with radiation therapy for patients with B-cell lymphoma (NCT02254772). Theoretically, intratumoral immune checkpoint antibodies can apply to any primary tumor that is accessible for intratumoral injection. To treat the metastatic tumor, however, intratumoral immune checkpoint antibodies should be able to induce systemic antitumor immunity. In animal studies, intratumoral immune checkpoint antibodies have shown antitumor immunity to the distant tumor, which is known as the abscopal effect (W. J. M. Mulder, S. Gnjatic, Nat Nanotechnol 12(9) (2017) 840-1; M. Bilusic, J. L. Gulley, Editorial: Local Immunotherapy: A Way to Convert Tumors From “Cold” to “Hot”, J Natl Cancer Inst 109(12) (2017); M. A. Aznar, et al., J Immunol 198(1) (2017) 31-9; A. Marabelle, et al., Ann Oncol 28(suppl_12) (2017) xii33-43; and V. Murthy, J. Minehart, D. H. Sterman, J Natl Cancer Inst 109(12) (2017)). But abscopal immunity is rarely described in patients except for a few cases in the context of ipilimumab, radiotherapy, and DC-based vaccination (M.A. Postow, et al., N Engl J Med 366(10) (2012) 925-31; E. F. Stamell, et al., Int J Radiat Oncol Biol Phys 85(2) (2013) 293-5; and J. Karbach, et al., Cancer Immunol Res 2(5) (2014) 404-9). Therefore, abscopal immunity can be considered as an important parameter to be observed in future clinical trials. Alternatively, the combination of intratumoral and intravenous immune checkpoint antibodies is applied to treat the metastatic tumor. For example, a phase I/II study is currently testing intratumoral ipilimumab plus intravenous nivolumab in patients with metastatic melanoma (NCT02857569).

Although many preclinical and clinical studies are adopting this treatment, some challenges of local injection of immune checkpoint antibodies remain. First, the retention time of antibodies at local sites is short. For example, after subcutaneous injection, the retention time of IgG at the injection site was about 6.8 hours (F. Wu, et al., Pharm Res 29(7) (2012) 1843-53)). The short retention time requires frequent local injections, which may lead to clinical inconvenience and low patient compliance (D. Schweizer, et al., Controlled release of therapeutic antibody formats, Eur J Pharm Biopharm 88(2) (2014) 291-309). Second, locally injected antibodies would eventually enter into the blood circulation. It is estimated that the systemic exposure of subcutaneously injected antibodies was about 50-80% of that of intravenously infused antibodies (W. F. Richter, B. Jacobsen, Drug Metab Dispos 42(11) (2014) 1881-9). The high systemic exposure of locally injected antibodies renders a high risk of side effects (J. Ishihara, et al., Sci Transl Med 9(415) (2017)).

A controlled release system is needed for local antibody injection to solve those challenges. Such a system could be able to increase local retention time and decrease the systemic exposure of antibodies. In addition, the system should be convenient for local injection. Also, the system should be adjustable to control the release of antibodies. To develop such a system as described herein, immune tolerant elastin-like polypeptides (iTEPs) were used as a carrier to deliver antibodies. iTEPs have the phase transition property that is related to its transition temperature (Tt). iTEPs are soluble in aqueous solution when the temperature is below Tt, and become insoluble and precipitate from the solution when the temperature is above Tt (P. Wang, et al., Biomaterials 182 (2018) 92-103). For example, if the Tt of an iTEP is below body temperature, the iTEP would precipitate and form depots after being injected into the body. The polypeptide or iTEP depots are released slowly, residing at the injection sites up to weeks (M. Amiram, et al., J Control Release 172(1) (2013) 144-51; S. M. Sinclair, et al., J Control Release 171(1) (2013) 38-47; M. Amiram, et al., Proc Natl Acad Sci USA 110(8) (2013) 2792-7; and K.M. Luginbuhl, et al., Nat Biomed Eng 1 (2017)). If antibodies are linked to those depots, the antibodies are expected to release slowly from the injection sites. In order to link iTEP(s) to antibodies (e.g., IgGs), an IgG binding domain (IBD) was attached to an iTEP to generate a recombinant polypeptide (can be referred to as an iTEP-IBD). “IBD” refers to a protein domain deriving from protein G (B. Guss, et al., EMBO J 5(7) (1986) 1567-75; and A. M. Gronenborn, G. M. Clore, ImmunoMethods 2(1) (1993) 3-8). IBD can bind to IgG with a high affmity of about 10 nM (M. Hutt, et al., J Biol Chem 287(7) (2012) 4462-9; and F. Unverdorben, et al., PLoS One 10(10) (2015) e0139838). As disclosed herein, the results show that a mixture of the recombinant proteins disclosed herein (e.g., iTEP-IBD) and IgG can form depots and trap IgG at injection sites, slowing down the release of IgG. The results also show that the release rate of IgG can be fine-tuned by controlling the molecular weight (MW) and the structure of the recombinant proteins disclosed herein (e.g. iTEP-IBD). Further, the recombinant protein (e.g., iTEP-IBD) was shown to reduce the systemic exposure of locally injected IgG. Finally, the results demonstrated that the recombinant protein (e.g. iTEP-IBD) could retain antibodies in the tumor. Taken together, these results described herein demonstrate the application of the disclosed recombinant polypeptides (e.g. iTEP-IBD) for local antibody administration.

iTEPs are proteins. iTEPs can self-assemble into nanoparticles (NPs) of a similar size. Disclosed here are compositions and methods using iTEPs (also referred to herein homologous amino acid repeat sequences) nanoparticles as drug delivery vehicles. In some aspects, the iTEPs disclosed herein can form a nanoparticle. In some aspects, the iTEPs disclosed herein will not form a nanoparticle. Whether a given iTEP as disclosed herein will form a nanoparticle can be dependent on a variety of factors including but not limited to the length of the iTEP (e.g., homologous amino acid repeat sequence), the hydrophobicity/hydrophilicity, or the composition of the diblock polymer, etc.

The iTEPs disclosed herein possess the desired transition property and were also tolerated by mouse humoral immunity. Also described herein, are two paired iTEPs that were opposite in hydrophobicity to make an amphiphilic diblock copolymer or fusion protein. A fusion protein can be generated by fusing two or more proteins together. The diblock copolymer can used to describe the fusion of two different iTEPs. The copolymer (e.g., fusion protein self-assembled into a NP. For example, SEQ ID NO: 1 and SEQ ID NO: 2 can be fused together to form a diblock polymer. In some aspects, the diblock polymer can then be fused or covalently bounded to an IgG binding domain.

Compositions

Recombinant polypeptides. As used herein, the term “recombinant polypeptide” refers to a polypeptide generated by a variety of methods including recombinant techniques. The recombinant polypeptides disclosed herein can comprise one or more homologous amino acid repeat sequences (e.g., an iTEP) and an IgG binding domain. Disclosed herein are recombinant polypeptides. In some aspects, the recombinant polypeptides can comprise an homologous amino acid repeat sequence. In some aspects, the homologous amino acid repeat sequence can have at least 75% amino acid sequence identity to the homologous amino acid repeat sequence. In some aspects, the homologous amino acid repeat sequence can be: Gly-Val-Leu-Pro-Gly-Val-Gly (SEQ ID NO: 1); Gly-Ala-Gly-Val-Pro-Gly (SEQ ID NO: 2); Val-Pro-Gly-Phe-Gly-Ala-Gly-Ala-Gly (SEQ ID NO: 3); Val-Pro-Gly-Leu-Gly-Ala-Gly-Ala-Gly (SEQ ID NO: 4); Val-Pro-Gly-Leu-Gly-Val-Gly-Ala-Gly (SEQ ID NO: 5); Gly-Val-Leu-Pro-Gly-Val-Gly-Gly (SEQ ID NO: 6); Gly-Val-Leu-Pro-Gly (SEQ ID NO: 7); Gly-Leu-Val-Pro-Gly-Gly (SEQ ID NO: 8); Gly-Leu-Val-Pro-Gly (SEQ ID NO: 9); Gly-Val-Pro-Leu-Gly (SEQ ID NO: 10); Gly-Ile-Pro-Gly-Val-Gly (SEQ ID NO: 11); Gly-Gly-Val-Leu-Pro-Gly (SEQ ID NO: 12); Gly-Val-Gly-Val-Leu-Pro-Gly (SEQ ID NO: 14); or Gly-Val-Pro-Gly (SEQ ID NO: 15); and an IgG binding domain.

In some aspects, the recombinant polypeptide comprises amino acid sequence Gly-(Gly-Val-Leu-Pro-Gly-Val-Gly)₂₈-Gly-Gly (SEQ ID NO: 23); Gly-(Gly-Ala-Gly-Val-Pro-Gly)₇₀-Gly-Gly (SEQ ID NO: 24); Gly-(Val-Pro-Gly-Phe-Gly-Ala-Gly-Ala-Gly)₂₁-Gly-Gly (SEQ ID NO: 25); or Gly-(Val-Pro-Gly-Leu-Gly-Ala-Gly-Ala-Gly)96-Gly-Gly (SEQ ID NO: 26).

In some aspects, the recombinant polypeptides can further comprise two or more homologous amino acid repeat sequences that are the same. For example, the homologous amino acid sequence can be Gly-Val-Leu-Pro-Gly-Val-Gly (SEQ ID NO: 1) repeated contiguously between 20 and 200 times (e.g., (Gly-Val-Leu-Pro-Gly-Val-Gly)28 (SEQ ID NO: 13); (Gly-Val-Leu-Pro-Gly-Val-Gly)₅₆ (SEQ ID NO: 16); or (Gly-Val-Leu-Pro-Gly-Val-Gly)₁₁₂ (SEQ ID NO: 17).

In some aspects, the recombinant polypeptides can further comprise two or more homologous amino acid repeat sequences that are different. In some aspects, the homologous amino acid sequence can be the same sequence repeated between 20 and 200 times contiguously and fused to a different homologous amino acid sequence that can be repeated between 20 and 200 times.

In some aspects, the recombinant polypeptide comprises a diblock copolymer or a fusion protein. Diblock copolymers or fusion proteins comprise two or three homologous amino acid repeat sequences linked together by covalent bonds. In some aspects, the diblock polymers can be formed by fusing, for example, Gly-Val-Leu-Pro-Gly-Val-Gly (SEQ ID NO: 1) to Gly-Ala-Gly-Val-Pro-Gly (SEQ ID NO: 2). In some aspects, the diblock polymer can be (SEQ ID NO: 1)x-(SEQ ID NO: 2)y or (SEQ ID NO: 2)y-(SEQ ID NO: 1)x, wherein x and y can be any number between 20-120, wherein any number between 20 and 120 indicates the number of times the respective homologous amino acid sequence is repeated.

In some aspects, one or more cysteine amino acid residues can be inserted between the diblock copolymer or fusion protein and an IgG binding domain. In some aspects, the number of cysteine amino acid residues can be 1, 2, 3, 4, 5, 10, 15, 20, 30, 40 or more or any number in between. In some aspects, the number of cysteine amino acid residues can be four. In some aspects, the cysteine amino acid residues can be separated by one or more glycine amino acid residues. The number of glycine amino acid residues can vary and depend on the number of cysteine amino acid residues inserted between the diblock copolymer and IgG binding domain. In some aspects, the number of glycine amino acid residues can be 1, 2, 3, 4, 5, 10, 15, 20, 30, 40 or more or any number in between. In some aspects, the number of glycine amino acid residues can be eight. For example, when four cysteine residues are inserted between the diblock copolymer and the IgG binding domain, eight glycine amino acid residues can be inserted to separate the adjacent cysteine amino acid residues. In some aspects, the diblock copolymers or fusion proteins can be amphiphilic. In some aspects, the diblock copolymers or fusion proteins can be fused with an IgG binding domain.

Also described herein, are recombinant polypeptides comprising an amino acid sequence conforming to the formula: Val-Pro-Gly-Xaa₁-Gly-Xaa₂-Gly-Ala-Gly wherein Xaa₁ is Leu or Phe and Xaa₂ is Ala or Val (SEQ ID NOs: 16-19), wherein the amino acid sequence is repeated.

In some aspects, the recombinant polypeptides described herein can further comprise one or more amino acid residues positioned at the N-terminus, C-terminus, or both the N-terminus and C-terminus of the recombinant polypeptide. In some aspects, the one or more amino acid residues can be glycine, alanine or serine or a combination thereof. In some aspects, the recombinant polypeptides can comprise the amino acid sequence Gly-(Val-Pro-Gly-Phe-Gly-Ala-Gly-Ala-Gly)₂₁-Gly-Gly (SEQ ID NO: 25); or Gly-(Val-Pro-Gly-Leu-Gly-Ala-Gly-Ala-Gly)₉₆- Gly-Gly (SEQ ID NO: 26); or XX-(Val-Pro-Gly-Leu-Gly-Val-Gly-Ala-Gly)_{X}-XX (SEQ ID NO: 27). As described below, “XX” can be one or more glycine amino acid residues at both the C-terminus and the N-terminus ends; and “x” can be 2, 3, 4, 5, 10, 15, 20, 30, 40, 50, 100, 150, 200 or any number in between. SEQ ID NO: 27 serves as an example of a homologous amino acid repeat sequence that is repeated “x” number of times, and is flanked by one or more glycine amino acid residues at both the C-terminus and the N-terminus ends. Any of the homologous amino acid sequences can be flanked by one or more glycine amino acid residues at either the C-terminus, the N-terminus, or both, and the number of glycine amino acids residues at either the C-terminus, the N-terminus, or both can be 2, 3, 4, 5, 10, 15, 20, 30, 40, 50, 100, 150, 200 or any number in between.

In some aspects, the identified molecular weight of the recombinant polypeptide can be between 10 and 100 kDa. In some aspects, the identified molecular weight of the recombinant polypeptide can be between 20 and 100 kDa.

Homologous amino acid repeat. As used herein, the term “homologous amino acid repeat” or “homologous amino acid repeat sequence” or “monomer” refers to an amino acid sequence comprising any of the 20 protein amino acids and is reiterated or duplicated linearly. Also, as used herein, the term “homologous amino acid sequence repeat” can refer to an iTEP sequence. In some aspects, the homologous amino acid repeat sequence can be repeated. The homologous amino acid repeat sequence can be repeated 2, 3, 4, 5, 10, 15, 20, 30, 40, 50, 100, 150, 200 times or more or any number of times in between. In some aspects, the homologous amino acid repeat can be repeated no more than 100 times. In some aspects, the homologous amino acid repeat can be repeated no more than 200 time. In another aspect, the homologous amino acid repeat can be repeated at least 20 times. In some aspects, the homologous amino acid repeat sequence can be repeated between 20 and 30 times, 30 and 40 times, 40 and 50 times, 50 and 60 times, 60 and 70 times, 70 and 80 times, 80 and 90 times, 90 and 100 times, 100 and 110 times, or 110 and 120 times.

In some aspects, the homologous amino acid repeat sequence can be the sequence Gly-Val-Leu-Pro-Gly-Val-Gly (SEQ ID NO: 1); Gly-Ala-Gly-Val-Pro-Gly (SEQ ID NO: 2); Val-Pro-Gly-Phe-Gly-Ala-Gly-Ala-Gly (SEQ ID NO: 3); Val-Pro-Gly-Leu-Gly-Ala-Gly-Ala-Gly (SEQ ID NO: 4); Val-Pro-Gly-Leu-Gly-Val-Gly-Ala-Gly (SEQ ID NO: 5); Gly-Val-Leu-Pro-Gly-Val-Gly-Gly (SEQ ID NO: 6); Gly-Val-Leu-Pro-Gly (SEQ ID NO: 7); Gly-Leu-Val-Pro-Gly-Gly (SEQ ID NO: 8); Gly-Leu-Val-Pro-Gly (SEQ ID NO: 9); Gly-Val-Pro-Leu-Gly (SEQ ID NO: 10); Gly-Ile-Pro-Gly-Val-Gly (SEQ ID NO: 11); Gly-Gly-Val-Leu-Pro-Gly (SEQ ID NO: 12); Gly-Val-Gly-Val-Leu-Pro-Gly (SEQ ID NO: 14); or Gly-Val-Pro-Gly (SEQ ID NO: 15). In some aspects, the homologous amino acid repeat sequence can be the sequence Gly-Val-Leu-Pro-Gly-Val-Gly (SEQ ID NO: 1). In some aspects, the homologous amino acid repeat sequence can be the sequence Gly-Ala-Gly-Val-Pro-Gly (SEQ ID NO: 2). Table 1 lists examples of homologous amino acid repeat sequences.

TABLE 1
Homologous Amino Acid Repeat Sequences

| SEQ ID NO: | Homologous Amino Acid Repeat |
|---|---|
| 1 | Gly-Val-Leu-Pro-Gly-Val-Gly |
| 2 | Gly-Ala-Gly-Val-Pro-Gly |
| 3 | Val-Pro-Gly-Phe-Gly-Ala-Gly-Ala-Gly |
| 4 | Val-Pro-Gly-Leu-Gly-Ala-Gly-Ala-Gly |
| 5 | Val-Pro-Gly-Leu-Gly-Val-Gly-Ala-Gly |
| 6 | Gly-Val-Leu-Pro-Gly-Val-Gly-Gly |
| 7 | Gly-Val-Leu-Pro-Gly |
| 8 | Gly-Leu-Val-Pro-Gly-Gly |
| 9 | Gly-Leu-Val-Pro-Gly |
| 10 | Gly-Val-Pro-Leu-Gly |
| 11 | Gly-Ile-Pro-Gly-Val-Gly |
| 12 | Gly-Gly-Val-Leu-Pro-Gly |
| 14 | Gly-Val-Gly-Val-Leu-Pro-Gly |
| 15 | Gly-Val-Pro-Gly |

In some aspects, the homologous amino acid repeat sequence can be the sequence (Gly-Val-Leu-Pro-Gly-Val-Gly)₂₈ (SEQ ID NO: 13); (Gly-Val-Leu-Pro-Gly-Val-Gly)₅₆ (SEQ ID NO: 16); or (Gly-Val-Leu-Pro-Gly-Val-Gly)₁₁₂ (SEQ ID NO: 17).

In another aspect, the homologous amino acid repeat sequence is not the amino acid sequence: Gly-Gly-Val-Pro-Gly (SEQ ID NO: 28).

In some aspects, the homologous amino acid repeat sequence can comprise four or more amino acid residues. In some aspects, no more than one proline can be present in a homologous amino acid repeat sequence. The homologous amino acid repeat sequence can exist as a naturally occurring sequence in an elastin. The homologous amino acid repeat sequence can also be naturally flanked by one or more glycine residues at both the N-terminus and C-terminus ends.

In some aspects, the homologous amino acid repeat can be elastin-derived. The homologous amino acid repeat sequence can be derived from a mouse and/or human elastin. In some aspects, the homologous amino acid repeat sequence can be derived from a mouse and/or human elastin that can be further flanked by one or more glycine residues at both the C-terminus and the N-terminus ends.

In some aspects, the homologous amino acid repeat can exhibit a certain degree of identity or homology to the homologous amino acid repeat, and wherein the homologous amino acid repeat can be one or more of SEQ ID NOs: 1-12, 14 and 15, etc. The degree of identity can vary and be determined by methods known to one of ordinary skill in the art. The terms “homology” and “identity” each refer to sequence similarity between two polypeptide sequences. Homology and identity can each be determined by comparing a position in each sequence which can be aligned for purposes of comparison. When a position in the compared sequence is occupied by the same amino acid residue, then the polypeptides can be referred to as identical at that position; when the equivalent site is occupied by the same amino acid (e.g., identical) or a similar amino acid (e.g., similar in steric and/or electronic nature), then the molecules can be referred to as homologous at that position. A percentage of homology or identity between sequences is a function of the number of matching or homologous positions shared by the sequences. The homologous amino acid repeat sequence of a recombinant polypeptide described herein can have at least or about 25%, 50%, 65%, 75%, 80%, 85%, 90%, 95%, 96%, 97%, 98%, or 99% identity or homology to the homologous amino acid repeat sequence, and wherein the homologous amino acid repeat sequence can be one or more of SEQ ID NOs: 1-12, 14, and 15 (for example, see, Table 1).

In some aspects, the recombinant polypeptide described herein can further comprise one or more amino acid residues positioned at the N-terminus, C-terminus, or both the N-terminus and C-terminus of the recombinant polypeptide. The one or more amino acid residues can be glycine, alanine or serine or a combination thereof. In some aspects, the one or more amino acid residues positioned at the N-terminus, C-terminus, or both the N-terminus and C-terminus of the recombinant polypeptide can be any amino acid residue that reduces immunogenicity.

IgG binding domain. Disclosed herein, are recombinant polypeptides comprising an IgG binding domain. In some aspects, the recombinant polypeptides can comprise at least one homologous amino acid repeat sequence that can be repeated at least two times covalently bound to an IgG binding domain.

In some aspects, the IgG binding domain of the disclosed recombinant polypeptides can be derived from protein G. In some aspects, the IgG binding domain can be a sequence that can bind to IgG1, IgG2, IgG3 or IgG4. As used herein, the term “derived from” can mean “come from” or “based on”. For example, the IgG binding domain sequence can be derived from a protein G sequence and be 75%, 80%, 85%, 90%, 95%, 96%, 97%, 98%, 99% or 100% same or be a variant or a fragment of the protein G base or original protein G sequence.

Disclosed herein are IgG binding domains comprising the sequence or is at least 75% identical to the amino acid sequence

(SEQ ID NO: 18)
TTYKLVINGKTLKGETTTKAVDAETAEK
AFKQYANDNGVDGVWTYDDATKTFTVTE.
In some aspects, the IgG binding domain
can comprise the sequence
(SEQ ID NO: 18)
TTYKLVINGKTLKGETTTKAVDAETAEK
AFKQYANDNGVDGVWTYDDATKTFTVTE,
or a fragment or a variant thereof.
In some aspects, the variant can be:
(SEQ ID NO: 19)
TTYKLILNGKTLKGETTTEAVDAATAEK
VFKQYANDNGVDGEWTYDDATKTFTVTE;
(SEQ ID NO: 20)
TTYKLVINGKTLKGETTTEAVDAATAEK
VFKQYANDNGVDGEWTYDDATKTFTVTE;
(SEQ ID NO: 21)
TTYKLVINGKTLKGETTTKAVDAETAAA
AFAQYANDNGVDGVWTYDDATKTFTVTE;
(SEQ ID NO: 22)
TTYKLVINGKTLKGETTTKAVDAETAAA
AFAQYARRNGVDGVWTYDDATKTFTVTE;
or
(SEQ ID NO: 29)
TTYKLVIAGKTLKGETTTEAVDAATAEK
VFKQYANDAGVDGEWTYDDATKTFTVTE
or a fragment or a variant thereof.
In some aspects, the fragment can be:
(SEQ ID NO: 30)
TTEAVDAATAEKVFKQYANDNGVDGEWTYDDATKTFTVTE;
(SEQ ID NO: 31)
QYANDNGVDGEWTYDDATKTFTVTE;
(SEQ ID NO: 32)
EKVFKQYANDNGVDGEWTY;
or
(SEQ ID NO: 33)
NDNGVDGEWTY.

Linkers. The recombinant polypeptides described herein can further comprise one or more linkers. A given linker within the compositions or recombinant polypeptides disclosed herein can provide a cleavable linkage (e.g., a thioester linkage). Sites available for linking can be identified on the recombinant polypeptides described herein. In some aspects, linkers in the disclosed recombinant polypeptides can comprise a group that is reactive with a primary amine on the recombinant polypeptide to which an IgG binding domain can be attached (e.g., via conjugation). Useful linkers are available from commercial sources. In some aspects, the linker can be 4-(4-N-maleimidophenyl)butyric acid hydrazide hydrochloride (MPBH). One of ordinary skill in the art is capable of selecting an appropriate linker.

The linker can be attached to the disclosed recombinant polypeptides via a covalent bond. To form covalent bonds, a chemically reactive group can be used, for instance, that has a wide variety of active carboxyl groups (e.g., esters) where the hydroxyl moiety is physiologically acceptable at the levels required to modify the recombinant polypeptide.

In some aspects, the one or more linker sequences can be a peptide. In some aspects, the linker sequences can be repeated linearly and contiguously. For example, the linker sequence can be repeated 2, 3, 4, 5, 6, 7, 8, 9, 10, 11, 12, 13, 14, 15, 16, 17, 18, 19, or 20 times. In some aspects, the linker sequence can be GGGGS (SEQ ID NO: 34). In some aspects, the linker sequence can be GGGGC (SEQ ID NO: 35). In some aspects, the linker sequence can be located between the homologous amino acid repeat sequence and the IgG binding domain. For example, from the N-terminus to the C-terminus, a recombinant polypeptide can comprise: a homologous amino acid repeat sequence (e.g., SEQ ID NO: 1) covalently bound to a linker sequence which can be covalently bound to the IgG binding domain; or IgG binding domain covalently bound to a linker sequence which can be covalently bound to a homologous amino acid repeat sequence (e.g., SEQ ID NO: 1).

In some aspects, the recombinant polypeptide can comprise any one of the amino acid sequences: (GVLPGVG)₂₈-GGGGS-TTYKLVINGKTLKGETTTKAVDAETAEKAFKQYANDNGVDGVWTYDDATKTFTVT E (SEQ ID NO: 36); (GVLPGVG)₅₆-GGGGS-TTYKLVINGKTLKGETTTKAVDAETAEKAFKQYANDNGVDGVWTYDDATKTFTVT E (SEQ ID NO: 37); or (GVLPGVG)₁₁₂-GGGGS-TTYKLVINGKTLKGETTTKAVDAETAEKAFKQYANDNGVDGVWTYDDATKTFTVT E (SEQ ID NO: 38).

In some aspects, the recombinant polypeptides disclosed herein can further comprise a second linker sequence. In some aspects, the second linker sequence can be (GGGGC)₄ (SEQ ID NO: 39). In some aspects the second linker must have a cysteine. The second linker can repeated from 1 to 20 times or any number in between. In some aspects, the second linker sequence can be located between a homologous amino acid repeat sequence and a first linker sequence. In some aspects, the (GGGGC)₄ (SEQ ID NO: 40) can be located between a homologous amino acid repeat sequence and a first linker sequence. In some aspects, the recombinant polypeptides described herein can be (GVLPGVG)₂₈-(GGGGC)₄-GGGGS-TTYKLVINGKTLKGETTTKAVDAETAEKAFKQYANDNGVDGVWTYDDATKTFTVT E (SEQ ID NO: 41); (GVLPGVG)₅₆-(GGGGC)₄-GGGGS-TTYKLVINGKTLKGETTTKAVDAETAEKAFKQYANDNGVDGVWTYDDATKTFTVT E (SEQ ID NO: 42); or (GVLPGVG)₁₁₂-(GGGGC)₄-GGGGS-TTYKLVINGKTLKGETTTKAVDAETAEKAFKQYANDNGVDGVWTYDDATKTFTVT E (SEQ ID NO: 43).

TABLE 2
Examples of sequences and molecular weight
(MW) of recombinant polypeptides.

| | | | SEQ |
|---|---|---|---|
| Poly- | Sequences (from N- | MW | ID |
| peptides | to C-terminus)^{a} | (kDa) | NO. |
| iTEP₂₈- | (GVLPGVG)₂₈-GGGGS- | 22.7 | 36 |
| IBD | TTYKLVINGKTLKGET | | |
| | TTKAVDAETAEKAFKQ | | |
| | YANDNGVDGVWTYDDA | | |
| | TKTFTVTE | | |
| iTEP₅₆- | (GVLPGVG)₅₆-GGGGS- | 38.9 | 37 |
| IBD | TTYKLVINGKTLKGET | | |
| | TTKAVDAETAEKAFKQY | | |
| | ANDNGVDGVWTYDDATK | | |
| | TFTVTE | | |
| iTEP₁₁₂- | (GVLPGVG)₁₁₂-GGGGS- | 71.4 | 38 |
| IBD | TTYKLVTNGKTLKGET | | |
| | TTKAVDAETAEKAFKQY | | |
| | ANDNGVDGVWTYDDATK | | |
| | TFTVTE | | |
| iTEP₂₈-C- | (GVLPGVG)₂₈-(GGGGC)₄- | 24.0 | 41 |
| IBD | GGGGS-TTYKLVIN | | |
| | GKTLKGETTTKAVDAE | | |
| | TAEKAFKQYANDNGVDGV | | |
| | WTYDDATKTFTVTE | | |
| iTEP₅₆-C- | (GVLPGVG)₅₆-(GGGGC)₄- | 40.3 | 42 |
| IBD | GGGGS-TTYKLVIN | | |
| | GKTLKGETTTKAVDAETAE | | |
| | KAFKQYANDNGVDGVWTYD | | |
| | DATKTFTVTE | | |
| iTEP₁₁₂-C- | (GVLPGVG)₁₁₂-(GGGGC)₄- | 72.7 | 43 |
| IBD | GGGGS-TTYKLVINGKTLKGE | | |
| | TTTKAVDAETAEKAFKQYAND | | |
| | NGVDGVWTYDDATKTFTVTE | | |

^{a} The subscripts after parentheses were the number of repeating sequences in the parentheses.
A “GGGGS” sequence (SEQ ID NO: 34) was inserted between before IBD to increase flexibility.

Therapeutic agent. Disclosed herein are recombinant polypeptides further comprising one or more therapeutic agents. A wide variety of therapeutic agents can be incorporated with, associated with, or linked to the recombinant polypeptides disclosed herein. A variety of therapeutic agents can be linked, bound (e.g., non-covalently) or associated with the recombinant polypeptide sequences described herein. In some aspects, the therapeutic agent can be incorporated into the recombinant polypeptides disclosed herein indirectly or directly. The therapeutic agents can be a peptide, an antibody or fragment thereof, an antibody-drug conjugate or an Fc-fusion protein. The therapeutic agents can also be a chemical compound, a protein, a peptide, a small molecule or a cell. Examples of therapeutic agents include but are not limited to peptide vaccines, antibodies, nucleic acids (e.g., siRNA) and cell-based agents (e.g., stem cells, CAR-T cells). In some aspects, the therapeutic agent can be an IgG or fragment thereof. In some aspects, one or more of the therapeutic agents can be an anti-cancer agent. The anti-cancer agent can be an antibody or fragment thereof or an antibody that is part of an antibody-drug conjugate or an Fc-fusion protein that has anti-cancer properties. In some aspects, the anti-cancer agent can be an anti-PD-1 antibody, anti-PD-L1 antibody or an anti-CTLA-4 antibody. In some aspects, the anti-PD-1 antibody can be nivolumab, pembrolizumab, or cemiplimab. In some aspects, the anti-PD-L1 antibody can be avelumab, durvalumab, or atezolizumab. In some aspects, the anti-CTLA-4 antibody can be ipilimumab. In some aspects, the anti-cancer agent can be an anti-cancer antibody or fragment thereof, an anti-cancer Fc-fusion or an anti-cancer antibody that can be part of an antibody drug-conjugate.-Examples of anti-cancer antibodies or fragments thereof include but are not limited to ofatumumab (anti-CD20), bevacizumab (anti-VEGF), blinatumumab (anti-CD3 and CD19), ramucirumab (anti-VEGFR2), daratumumab (anti-CD38), elotuzumab (anti-SLAMF7), cetuximab (anti-EGFR), obinutuzumab (anti-CD20), trastuzumab (anti-HER2), pertuzumab (anti-HER2), necitumumab (anti-EGFR), denosumab (anti-RANKL), rituximab (anti-CD20), siltuximab (anti-IL-6), dinutuximab (anti-GD2), panitumumab (anti-EGFR), and mogamulizumab (anti-CCR4). Examples of anti-cancer Fc-fusion protein also includes but are not limited to aflibercept. Examples of anti-cancer antibody that can be part of an antibody drug-conjugate include but are not limited to Gemtuzumab Ozogamicin, Brentuximab Vedotin, Ado-Trastuzumab Emtansine, Inotuzumab Ozogamicin, and Polatuzumab vedotin-piiq.

The recombinant polypeptides as described herein can also be used as a carrier for scaffolding materials, for example, for cell adherence and growth, and, thus, can be used in tissue repair or cell-based therapy. The recombinant polypeptides can also be used as a matrix gel, for example, to facilitate cell growth in vitro and in vivo; and as an adjuvant.

Methods of Making Recombinant Polypeptides

Disclosed herein are methods that can be used to produce the recombinant polypeptides described herein.

Design. In some aspects, the recombinant polypeptides comprising homologous amino acid repeat sequences (e.g., iTEPs) described herein can be designed as polymers of peptides derived from elastin. The recombinant polypeptides comprising homologous amino acid repeats sequences should be humorally tolerant in mice and humans. The recombinant polypeptides and the homologous amino acid repeat sequences selected should not intrinsically induce an autoimmune response (i.e., the sequences should not intrinsically bind to B cell or T cell receptors).

To reduce the possibility of generating recombinant polypeptides comprising homologous amino acid repeat sequences that are immunogenic, at least two strategies can be employed. First, common, existing peptide repeat sequences within human and mouse elastins can be used as a component of the homologous amino acid repeat sequence to limit generating extrinsic junction sequences. Second, when one or more extrinsic junction sequences were produced, the homologous amino acid repeat sequences should be four residues or longer and from elastins; and be flanked by one or more glycine residues at the N- and C-terminuses. By using homologous amino acid repeat sequences that are longer rather than shorter, the number of extrinsic junction sequences can be reduced. Reducing or eliminating extrinsic junction sequences may reduce the immunogenicity of the recombinant polypeptide or homologous amino acid repeat sequence.

In some aspects, for the homologous amino acid repeat sequences to have the phase transition property, they can be designed to have one proline amino acid residue and one or more valine amino acid residues.

The recombinant polypeptides disclosed herein can be produced by synthetic methods and recombinant techniques used routinely to produce proteins from nucleic acids or to synthesize polypeptides in vitro. The recombinant polypeptides and the homologous amino acid repeat sequence and/or diblock polymers can be stored in an unpurified or in an isolated or substantially purified form until later use.

In some aspects, the recombinant polypeptides disclosed herein can be a recombinant fusion protein or diblock polymer. In some aspects, the recombinant polypeptides can be expressed in a variety of expression systems (e.g., E.coli, yeast, insect cell, and mammalian cell cultures; and plants). Briefly, a plasmid DNA encoding the recombinant polypeptides can be transfected into cells of any of the expression systems described above. After the recombinant polypeptide (e.g., SEQ ID NO: 1-SEQ ID NO: 2) is produced in any one of these systems, they can then also be purified, lyophilized and stored until use.

The homologous amino acid repeat sequences described herein can be modified to chemically interact with, or to include, a linker as described herein. These recombinant polypeptides, homologous amino acid repeat sequences and peptide-linker constructs are within the scope of the present disclosure and can be packaged as a component of a kit with instructions for completing the process of attaching (e.g., conjugation) to an IgG binding domain and/or association with a therapeutic agent. The homologous amino acid repeat sequences can be modified to include a cysteine residue or other thio-bearing moiety (e.g., C—SH) at the N-terminus, C-terminus, or both.

In some aspects, the therapeutic agent (e.g., an IgG or antibody) can be mixed with the recombinant polypeptide using methods known to one of ordinary skill in the art. For example, the therapeutic agent (e.g., an antibody) and the recombinant polypeptide (e.g., iTEP-IBD) can be mixed together in solution in a container such as a tube through pipetting, tapping, shaking, vortexing or other methods.

Configurations. The disclosed recombinant polypeptides, including the homologous amino acid repeat sequences, number of times the homologous amino acid repeat sequence is repeated, the IgG binding domain, linker(s), and therapeutic agent can be selected independently. One of ordinary skill in the art would understand that the component parts need to be associated in a compatible manner. As disclosed herein, the recombinant polypeptides disclosed herein can be used to deliver therapeutic agents to a patient for the treatment of cancer and autoimmune diseases. In some aspects, a therapeutic agent can be conjugated to a recombinant polypeptide. In some aspects, the recombinant polypeptide can comprise a homologous amino acid repeat sequence covalently linked to an IgG binding domain. In some aspects, the therapeutic agent can be non-covalently conjugated to the IgG binding domain. The number of therapeutic agents per recombinant polypeptide can be controlled by adding additional IgG binding domains. One IgG binding domain can be bound (e.g., non-covalently) to one therapeutic agent. In some aspects, the recombinant polypeptide can comprise one or more or two or more IgG binding domains. As such, the recombinant polypeptide can comprise two or more therapeutic agents. For example, the linear configuration of a recombinant polypeptide comprising two IgG binding domains can be: IBD-iTEP-IBD, iTEP-IBD-iTEP-IBD or IBD-iTEP-IBED-iTEP. In some aspects, the iTEP can be any of the homologous amino acid repeat sequences disclosed herein. In some aspects, the homologous amino acid repeat sequences can be the same or different. IN some aspects, the IBD can comprise any of the sequences disclosed herein. In some aspects, the IBD can comprise the same or a different sequence. For example, one or more cysteines amino acid residues can be added at one of end of a homologous amino acid repeat sequence (e.g., iTEP) and be used as conjugation sites for one or more IgG binding domains. For example, eight cysteine residues can be added and provide eight conjugation sites for eight IgG binding domains. The therapeutic agents can be the same, different or any combination thereof. When two or cysteine residues are added to the end of a recombinant polypeptide as described herein, one or more spacers (e.g., glycine residues) can be inserted between, for example, two cysteine residues. The number of spacers can be adjusted according to the number of cysteine residues added or to the number of therapeutic molecules desired. The spacers serve to provide ample space to accommodate two or more IgG binding domains. Spacers can be one or more glycines or serines or a combination thereof. Alternatively, additional linker sequences can be incorporated into the recombinant polypeptide when more than one iTEP sequence and/or more than one IBD is present in the recombinant polypeptide.

Accordingly, in some aspects, the recombinant proteins and compositions disclosed herein can comprise one or more therapeutic agents. In some aspects, the recombinant polypeptide as described herein (e.g., an iTEP) and the therapeutic agent are present in a ratio of 1:1 (recombinant polypeptide:therapeutic agent). The recombinant polypeptide:therapeutic agent ratio can also be 2:2, 3:3, 4:4, 5:5, 6:6, 7:7, 8:8, 9:9, 10:10 or any other combinations thereof. The number of therapeutic agents that can be conjugated to the recombinant polypeptides described herein can be determined by the number of conjugation sites (e.g., IgG binding domains or cysteine residues) that are added in a given polypeptide. In some aspects, the recombinant polypeptide:therapeutic agent ratio can also be 0.5:1, 1:1, 2:1, 4:1, 8:1, 16:1, 24:1, 32:1 or any other combinations thereof. In some aspects, the recombinant polypeptide:therapeutic agent ratio can be between 0.5:1 (or alternatively, 1:2) and 32:1.

One or more cysteine residues can be added between to recombinant polypeptides described herein (e.g., between two iTEP molecules or two homologous amino acid repeat sequences). The cysteine residues can further be separated by adding two or more spacers (e.g., glycine residues). For example, four cysteine residues can be inserted between a diblock polymer (or copolymer or fusion protein) and an IgG binding domain. These cysteine residues, for instance, can be further separated by the addition of eight glycine residues.

Detectable labels. The recombinant polypeptides described herein can further comprise one or more labels or detection tags. (e.g., FLAG™ tag, epitope or protein tags, such as myc tag, 6 His, and fluorescent fusion protein). In some aspects, the label (e.g., FLAG™ tag) can fused to the recombinant polypeptide. In some aspects, the disclosed methods and compositions further comprise a recombinant polypeptide, or a polynucleotide encoding the same. In various aspects, the recombinant polypeptide comprises at least one epitope-providing amino acid sequence (e.g., “epitope-tag”), wherein the epitope-tag is selected from i) an epitope-tag added to the N- and/or C-terminus of the protein (e.g., recombinant polypeptide); or ii) an epitope-tag inserted into a region of the protein (e.g., recombinant polypeptide), and an epitope-tag replacing a number of amino acids in the protein (e.g., recombinant polypeptide). In some aspects, the detectable label can be referred to as a detectable moiety. In some aspects, the detectable label or detectable moiety can be covalently linked or covalently bound to the IgG binding domain. Also disclosed herein are methods of detecting a detectable moiety. The methods can comprise administering to the subject a therapeutically effective amount of the recombinant polypeptide as disclosed herein, wherein the IgG binding domain is covalently or non-covalently linked to a detectable moiety, thereby detecting the detectable moiety

Epitope tags are short stretches of amino acids to which a specific antibody can be raised, which in some aspects allows one to specifically identify and track the tagged protein that has been added to a living organism or to cultured cells. Detection of the tagged molecule can be achieved using a number of different techniques. Examples of such techniques include: immunohistochemistry, immunoprecipitation, flow cytometry, immunofluorescence microscopy, ELISA, immunoblotting (“Western blotting”), and affmity chromatography. Epitope tags add a known epitope (e.g., antibody binding site) on the subject protein, to provide binding of a known and often high-affinity antibody, and thereby allowing one to specifically identify and track the tagged protein that has been added to a living organism or to cultured cells. Examples of epitope tags include, but are not limited to, myc, T7, GST, GFP, HA (hemagglutinin), V5 and FLAG tags. The first four examples are epitopes derived from existing molecules. In contrast, FLAG is a synthetic epitope tag designed for high antigenicity (see, e.g., U.S. Pat. Nos. 4,703,004 and 4,851,341). Epitope tags can have one or more additional functions, beyond recognition by an antibody.

In some aspects, the disclosed methods, recombinant polypeptide and compositions comprise an epitope-tag wherein the epitope-tag has a length of between 6 to 15 amino acids. In an alternative aspect, the epitope-tag has a length of 9 to 11 amino acids. The disclosed methods and compositions can also comprise a recombinant polypeptide comprising two or more epitope-tags, either spaced apart or directly in tandem. Further, the disclosed methods and composition can comprise 2, 3, 4, 5 or even more epitope-tags, as long as the recombinant polypeptide maintains its biological activity/activities (e.g., “functional”).

In some aspects, the epitope-tag can be a VSV-G tag, CD tag, calmodulin-binding peptide tag, S-tag, Avitag, SF-TAP-tag, strep-tag, myc-tag, FLAG-tag, T7-tag, HA (hemagglutinin)-tag, His-tag, S-tag, GST-tag, or GFP-tag. The sequences of these tags are described in the literature and well known to the person of skill in art.

As described herein, the term “immunologically binding” is a non-covalent form of attachment between an epitope of an antigen (e.g., the epitope-tag) and the antigen-specific part of an antibody or fragment thereof. Antibodies are preferably monoclonal and must be specific for the respective epitope tag(s) as used. Antibodies include murine, human and humanized antibodies. Antibody fragments are known to the person of skill and include, amongst others, single chain Fv antibody fragments (scFv fragments) and Fab-fragments. The antibodies can be produced by regular hybridoma and/or other recombinant techniques. Many antibodies are commercially available.

The construction of recombinant polypeptides from domains of known proteins, or from whole proteins or proteins and peptides, is well known. Generally, a nucleic acid molecule that encodes the desired protein and/or peptide portions are joined using genetic engineering techniques to create a single, operably linked fusion oligonucleotide. Appropriate molecular biological techniques can be found in Sambrook et al. (Molecular Cloning: A laboratory manual Second Edition Cold Spring Harbor Laboratory Press, Cold spring harbor, N.Y., USA, 1989). Examples of genetically engineered multi-domain proteins, including those joined by various linkers, and those containing peptide tags, can be found in the following patent documents: U.S. Pat. No. 5,994,104 (“Interleukin-12 fusion protein”); U.S. Pat. No. 5,981,177 (“Protein fusion method and construction”); U.S. Pat. No. 5,914,254 (“Expression of fusion polypeptides transported out of the cytoplasm without leader sequences”); U.S. Pat. No. 5,856,456 (“Linker for linked fusion polypeptides”); U.S. Pat. No. 5,767,260 (“Antigen-binding fusion proteins”); U.S. Pat. No. 5,696,237 (“Recombinant antibody-toxin fusion protein”); U.S. Pat. No. 5,587,455 (“Cytotoxic agent against specific virus infection”); U.S. Pat. No. 4,851,341 (“Immunoaffinity purification system”); U.S. Pat. No. 4,703,004 (“Synthesis of protein with an identification peptide”); and WO 98/36087 (“Immunological tolerance to HIV epitopes”).

The placement of the functionalizing peptide portion (epitope-tag) within the subject recombinant polypeptides can be influenced by the activity of the functionalizing peptide portion and the need to maintain at least substantial recombinant polypeptide, such as TCR, biological activity in the fusion. Two methods for placement of a functionalizing peptide are: N-terminal, and at a location within a protein portion that exhibits amenability to insertions. Though these are not the only locations in which functionalizing peptides can be inserted, they serve as good examples, and will be used as illustrations. Other appropriate insertion locations can be identified by inserting test peptide encoding sequences (e.g., a sequence encoding the FLAG peptide) into a construct at different locations, then assaying the resultant fusion for the appropriate biological activity and functionalizing peptide activity, using assays that are appropriate for the specific portions used to construct the recombinant polypeptides. The activity of the subject recombinant polypeptides can be measured using any of various known techniques, including those described herein.

The methods disclosed herein related to the process of producing the recombinant polypeptides as disclosed herein can be readily modified to produce a pharmaceutically acceptable salt of the recombinant polypeptides. Pharmaceutical compositions including such salts and methods of administering them are within the scope of the present disclosure.

Pharmaceutical Compositions

As disclosed herein, are pharmaceutical compositions, comprising the recombinant polypeptides disclosed herein. Also disclosed herein, are pharmaceutical compositions, comprising a recombinant polypeptide(s) and a pharmaceutical acceptable carrier. In some aspects, the therapeutic agent can be an anti-cancer agent or an agent that can be used to treat an autoimmune disease. In some aspects, the therapeutic agent can be an antibody or fragment thereof, an antibody that is part of an antibody-drug conjugate or an Fc-fusion protein. In some aspects, the pharmaceutical composition can be formulated for parenteral administration, subcutaneous administration or direct injection. In some aspects, administration by injection can encompass directly administering any of the compositions disclosed herein including any of the recombinant polypeptides (including recombinant polypeptides non-covalently bound to a therapeutic agent) to one or more disease sites (e.g., a tumor). The compositions of the present disclosure also contain a therapeutically effective amount of a recombinant polypeptide as described herein. The compositions can be formulated for administration by any of a variety of routes of administration, and can include one or more physiologically acceptable excipients, which can vary depending on the route of administration. As used herein, the term “excipient” means any compound or substance, including those that can also be referred to as “carriers” or “diluents.” In some aspects, the compositions and recombinant polypeptides disclosed herein can further comprise a natural polymer, adjuvant, excipient, preservative, agent for delaying absorption, filler, binder, absorbent, buffer, or a combination thereof Preparing pharmaceutical and physiologically acceptable compositions is considered routine in the art, and thus, one of ordinary skill in the art can consult numerous authorities for guidance if needed.

The pharmaceutical compositions as disclosed herein can be prepared for oral or parenteral administration. Pharmaceutical compositions prepared for parenteral administration include those prepared for intravenous (or intra-arterial), intramuscular, subcutaneous, intraperitoneal, transmucosal (e.g., intranasal, intravaginal, or rectal), or transdermal (e.g., topical) administration. Aerosol inhalation can also be used to deliver the recombinant polypeptides. Thus, compositions can be prepared for parenteral administration that includes recombinant polypeptides dissolved or suspended in an acceptable carrier, including but not limited to an aqueous carrier, such as water, buffered water, saline, buffered saline (e.g., PBS), and the like. One or more of the excipients included can help approximate physiological conditions, such as pH adjusting and buffering agents, tonicity adjusting agents, wetting agents, detergents, and the like. Where the compositions include a solid component (as they may for oral administration), one or more of the excipients can act as a binder or filler (e.g., for the formulation of a tablet, a capsule, and the like). Where the compositions are formulated for application to the skin or to a mucosal surface, one or more of the excipients can be a solvent or emulsifier for the formulation of a cream, an ointment, and the like. Any of the compositions disclosed herein can be administered such that the composition changes to a depot after injection. For example, before an injection, any of the compositions disclosed herein (e.g., the recombinant polypeptides disclosed herein including the therapeutic agents) can be in a soluble solution. After the injection, for example, into a tissue, the composition can change and form a depot. The depot that can be formed can retain the therapeutic agent in the tissue longer compared to the administration of the therapeutic agent alone.

The pharmaceutical compositions can be sterile and sterilized by conventional sterilization techniques or sterile filtered. Aqueous solutions can be packaged for use as is, or lyophilized, the lyophilized preparation, which is encompassed by the present disclosure, can be combined with a sterile aqueous carrier prior to administration. The pH of the pharmaceutical compositions typically will be between 3 and 11 (e.g., between about 5 and 9) or between 6 and 8 (e.g., between about 7 and 8). The resulting compositions in solid form can be packaged in multiple single dose units, each containing a fixed amount of the above-mentioned agent or agents, such as in a sealed package of tablets or capsules. The composition in solid form can also be packaged in a container for a flexible quantity, such as in a squeezable tube designed for a topically applicable cream or ointment.

Methods of Treatment

Disclosed herein, are methods of treating a patient with cancer, the method comprising: administering to the patient a therapeutically effective amount of the pharmaceutical composition comprising any of the recombinant polypeptides disclosed herein.

Disclosed herein, are methods of treating a patient with cancer, the method comprising: (a) identifying a patient in need of treatment; and (b) administering to the patient a therapeutically effective amount of the pharmaceutical composition comprising any of the recombinant polypeptides disclosed herein..

Disclosed herein, are methods of treating a patient with an autoimmune disease, the method comprising: administering to the patient a therapeutically effective amount of the pharmaceutical composition comprising any of the recombinant polypeptides disclosed herein. Disclosed herein, are methods of treating a patient with an autoimmune disease, the method comprising: (a) identifying a patient in need of treatment; and (b) administering to the patient a therapeutically effective amount of the pharmaceutical composition comprising any of the recombinant polypeptides disclosed herein.

Disclosed herein are methods of treating a subject with cancer. Disclosed herein are methods of treating a subject with an autoimmune disease. Disclosed herein are methods of treating any disease or disorder in which the therapeutic agent to be administered to the subject with the disease or disorder is an antibody or fragment thereof. In some aspects, the diseases or disorders can include but are not limited to inflammation, autoimmune diseases, infectious diseases, blood diseases, cardiovascular diseases, metabolic diseases, bone diseases, muscle diseases, pain, ophthalmologic diseases, etc.

In some aspects, the methods can comprise administering to the subject a therapeutically effective amount of the pharmaceutical composition disclosed herein. In some aspects, the method can further comprise identifying a subject in need of treatment prior to the administering step.

Disclosed herein are methods of reducing tumor size in a subject in need thereof. In some aspects, the methods can comprise administering to the subject an effective amount of a composition comprising any of the recombinant polypeptides disclosed herein. In some aspects, the IgG binding domain can be non-covalently bound to a therapeutic agent, thereby reducing tumor size. In some aspects, the tumor can be a malignant tumor. In some aspects, the malignant tumor can be breast cancer, ovarian cancer, lung cancer, colon cancer, gastric cancer, head and neck cancer, glioblastoma, renal cancer, cervical cancer, peritoneal cancer, kidney cancer, pancreatic cancer, brain cancer, spleen cancer, prostate cancer, urothelial carcinoma, skin cancer, myeloma, lymphoma, or a leukemia.

Also disclosed herein are methods of administering to a subject a therapeutic agent conjugated to a recombinant polypeptide. In some aspects, the recombinant polypeptide can comprise a homologous amino acid repeat sequence covalently linked to an IgG binding domain, wherein the therapeutic agent is non-covalently conjugated to the IgG binding domain. In some aspects, the conjugate can be administered by direct injection. In some aspects, at least one of: (i) the bioavailability of the therapeutic agent is greater; (ii) the half-life of the therapeutic agent is greater, (iii) the systemic toxicity of the therapeutic agent is less, in the subject when the therapeutic agent is administered to the subject in conjugated form as the conjugate as compared to the same amount of the therapeutic agent administered to the subject in the same way in unconjugated form.

Also disclosed herein are methods of increasing the efficacy of a therapeutic agent or increasing the half-life of a therapeutic agent in a subject. In some aspects, the methods can comprise administering to the subject a therapeutic agent non-covalently conjugated to a recombinant polypeptide, wherein the recombinant polypeptide comprises a homologous amino acid repeat sequence covalently linked to a IgG binding domain, wherein the therapeutic agent is non-covalently conjugated to the IgG binding domain, and wherein the conjugate is administered by direct injection, whereby the efficacy or half-life of the therapeutic agent can be increased. In some aspects, the conjugate can be directly injected into the site(s) of the tumor or cancer or disease.

In some aspects, the conjugate can be administered to the subject in a treatment-effective amount. In some aspects, the conjugate can be administered to the subject by parenteral injection. In some aspects, the conjugate can be administered to the subject subcutaneously. In some aspects, the in vivo efficacy of the therapeutic agent can be enhanced in the subject compared to the same amount of the therapeutic agent administered to the subject in an unconjugated form.

The pharmaceutical compositions described above can be formulated to include a therapeutically effective amount of any of the recombinant polypeptides disclosed herein. Therapeutic administration encompasses prophylactic applications. Based on genetic testing and other prognostic methods, a physician in consultation with their patient can choose a prophylactic administration where the patient has a clinically determined predisposition or increased susceptibility (in some cases, a greatly increased susceptibility) to a type of cancer or autoimmune disease.

The pharmaceutical compositions described herein can be administered to the subject (e.g., a human patient) in an amount sufficient to delay, reduce, or preferably prevent the onset of clinical disease. Accordingly, in some aspects, the patient or subject can be a human patient or subject. In therapeutic applications, compositions can be administered to a subject (e.g., a human patient) already with or diagnosed with cancer (or an autoimmune disease) in an amount sufficient to at least partially improve a sign or symptom or to inhibit the progression of (and preferably arrest) the symptoms of the condition, its complications, and consequences. An amount adequate to accomplish this is defined as a “therapeutically effective amount.” A therapeutically effective amount of a pharmaceutical composition can be an amount that achieves a cure, but that outcome is only one among several that can be achieved. As noted, a therapeutically effect amount includes amounts that provide a treatment in which the onset or progression of the cancer (or an autoimmune disease) is delayed, hindered, or prevented, or the cancer (or the autoimmune disease) or a symptom of the cancer (or the autoimmune disease) is ameliorated. One or more of the symptoms can be less severe. Recovery can be accelerated in an individual who has been treated. The therapeutically effective amount of one or more of the therapeutic agents present within the compositions described herein and used in the methods as disclosed herein applied to mammals (e.g., humans) can be determined by one of ordinary skill in the art with consideration of individual differences in age, weight, and other general conditions (as mentioned above),In some aspects, the cancer can be a primary or secondary tumor. In other aspects, the primary or secondary tumor can be within the patient's breast, lung, colon, ovary, head, neck, skin, gastrointestinal tract, cervix, kidney, pancreas, brain, spleen, prostate, urothelial, lymph nodes, blood, epithelial cells of the abdomen, bone marrow, immune cells (e.g., spleen, lymphocytes, thymus).

Disclosed herein, are methods of treating a patient with cancer. The cancer can be any cancer. In some aspects, the cancer can be a solid cancer. In some aspects, the solid cancer can be lung cancer, colon cancer, breast cancer, brain cancer, liver cancer, prostate cancer, spleen cancer, muscle cancer, ovarian cancer, pancreatic cancer, skin cancer, and melanoma In some aspects, the cancer can be breast cancer, ovarian cancer, lung cancer, colon cancer, gastric cancer, head and neck cancer, glioblastoma, renal cancer, cervical cancer, peritoneal cancer, kidney cancer, pancreatic cancer, brain cancer, spleen cancer, prostate cancer, urothelial carcinoma, skin cancer, myeloma, lymphoma, or a leukemia. In an aspect, the cancer can be metastatic.

Disclosed herein, are methods of treating a patient with an autoimmune disease. The autoimmune disease can be any autoimmune disease or disorder. In some aspects, the autoimmune disease or disorder can be non-Hodgkin's lymphoma, rheumatoid arthritis, chronic lymphocytic leukemia, multiple sclerosis, systemic lupus erythematosus, autoimmune hemolytic anemia, pure red cell aplasia, idiopathic thrombocytopenic purpura, Evans syndrome, vasculitis, bullous skin disorders, Type 1 diabetes mellitus, Sjogren's syndrome, Devic's disease, or Graves' disease ophthalmopathy.

Amounts effective for this use can depend on the severity of the cancer (or autoimmune disease) and the weight and general state and health of the subject, but generally range from about 0.05 pg to about 1000 mg (e.g., 1-15 mg/kg) of an equivalent amount of the recombinant polypeptide per dose per subject. Suitable regimes for initial administration and booster administrations are typified by an initial administration followed by repeated doses at one or more hourly, daily, weekly, or monthly intervals by a subsequent administration. For example, a subject can receive a recombinant polypeptide comprising a therapeutic agent in the range of about 0.05 pg to 1,000 mg equivalent dose as compared to unbound or free therapeutic agent(s) per dose one or more times per week (e.g., 2, 3, 4, 5, 6, or 7 or more times per week). For example, a subject can receive 0.1 μg to 2,500 mg (e.g., 2,000, 1,500, 1,000, 500, 100, 10, 1, 0.5, or 0.1 mg) dose per week. A subject can also receive a recombinant polypeptide as disclosed herein in the range of 0.1 μg to 3,000 mg per dose once every two or three weeks. A subject can also receive 2 mg/kg every week (with the weight calculated based on the weight of the recombinant polypeptide or any part or component of the immunogenic bioconjugate).

The total effective amount of the recombinant polypeptide in the pharmaceutical compositions disclosed herein can be administered to a mammal as a single dose, either as a bolus or by infusion over a relatively short period of time, or can be administered using a fractionated treatment protocol in which multiple doses are administered over a more prolonged period of time (e.g., a dose every 4-6, 8-12, 14-16, or 18-24 hours, or every 2-4 days, 1-2 weeks, or once a month). Alternatively, continuous intravenous infusions sufficient to maintain therapeutically effective concentrations in the blood are also within the scope of the present disclosure.

Because the recombinant polypeptides of the present disclosure can be stable in serum and the bloodstream and in some cases more specific, the dosage of the recombinant polypeptides including any individual component can be lower (or higher) than an effective dose or therapeutically effective amount of any of the individual components when unbound. Accordingly, in some aspects, the therapeutic agent (e.g., the anti-cancer agent) administered can have an increased efficacy or reduced side effects when administered as part of a (or bound to (e.g., non-covalently bound) recombinant polypeptide as compared to when the therapeutic agent (e.g., anti-cancer agent) is administered alone or not as part of (or not bound to) a recombinant polypeptide. In some aspects, the therapeutic agent can have an increased half-life when administered to the recombinant polypeptide (e.g., non-covalently bound) as compared to when the therapeutic agent is administered alone or not bound to the recombinant polypeptide.

In some aspects, the pharmaceutical compositions disclosed herein can be administered with (simultaneously, before or after) or combined with the administration of a second and different pharmaceutical composition or therapy. The second pharmaceutical composition or therapy can be dependent on the treatment regimen and the type and severity of the cancer or the type and severity of the autoimmune disease. In some aspects, the second pharmaceutical composition or therapy can be chemotherapy.

### EXAMPLES

### Example 1

### Immune Tolerant Elastin-Like Polypeptide (ITEP) for Sustained Local Delivery of Immune Checkpoint Antibodies

Abstract. To address the challenges associated with systemic administration of immune checkpoint antibodies, immune tolerant elastin-like polypeptide (iTEP)-based systems were developed to improve the local delivery of immune checkpoint antibodies. Due to the phase transition property of iTEPs, the thermosensitive delivery system can form slow releasing depots at injection sites. To link antibodies to the depots, an IgG binding domain (IBD) was fused to an iTEP. The results described herein demonstrate that the iTEP-IBD polypeptide can extend the release of antibodies and increase the retention time of the antibodies at local injection sites. By controlling the design of the iTEP-IBD polypeptide, the release half-life of the antibody can be fine-tuned within about 17.2 to about 74.9 hours. Using melanoma as the disease model, the results show that the iTEP-IBD polypeptide retained the antibodies in the tumor for more than 72 hours. Also, the iTEP-IBD polypeptide reduced the antibody exposure in other organs and blood circulation, thereby decreasing the risk of side effects. These results suggest that the iTEP-IBD polypeptide can be used as a platform for local delivery of immune checkpoint antibodies in subjects with cancer.

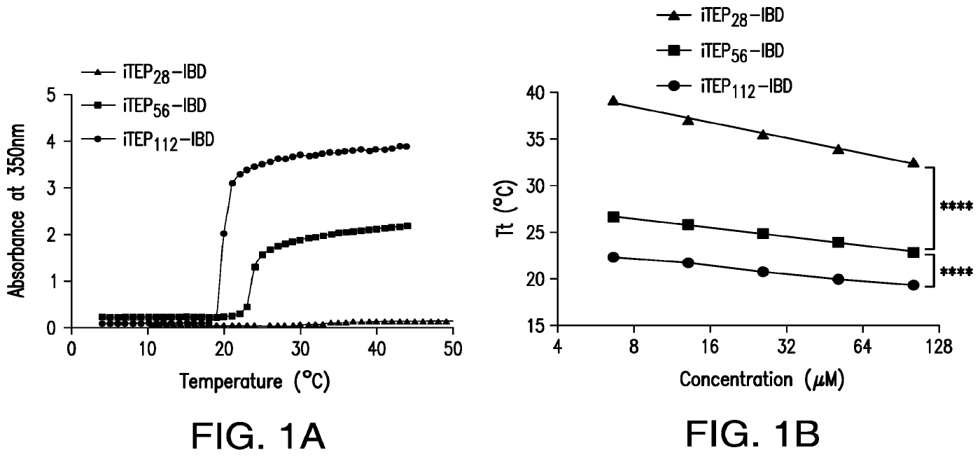

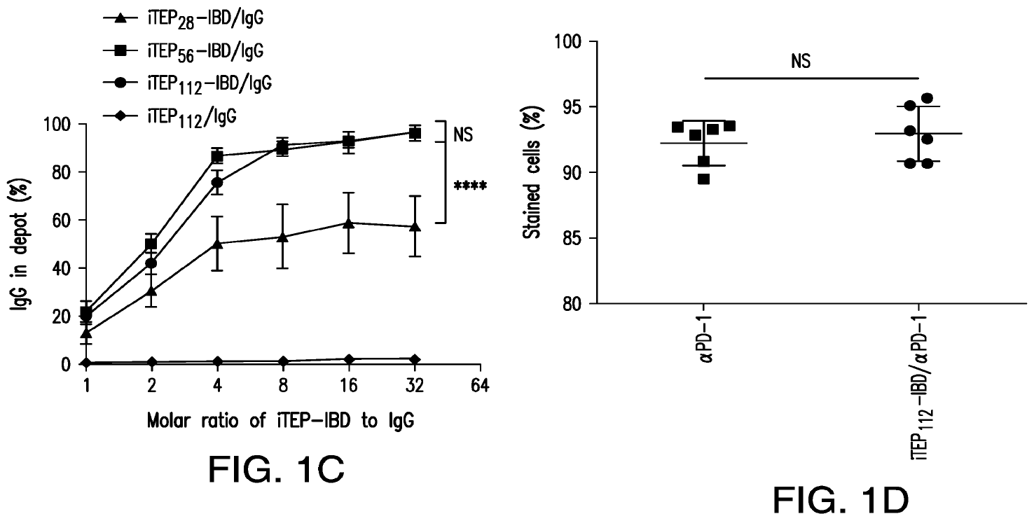

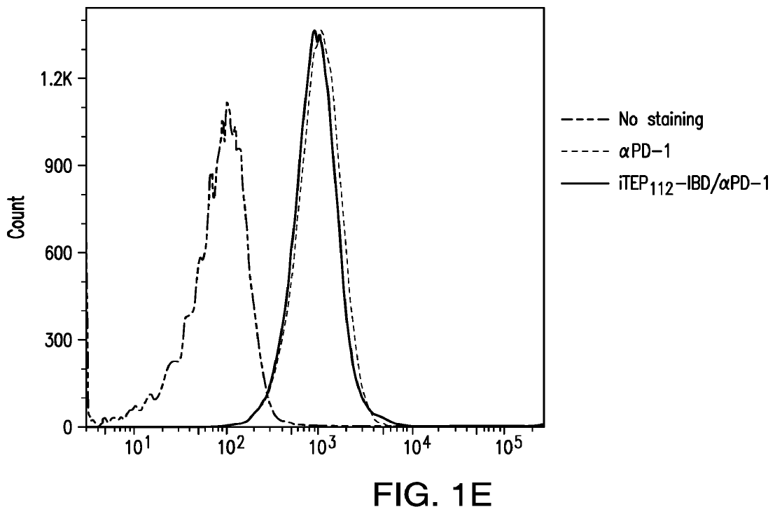

iTEP-IBD trapped IgG and did not impact the binding function of IgG. First IBD was fused to three different iTEPs with different molecular weight (MW): iTEP₂₈ (SEQ ID NO: 13), iTEP₅₆(SEQ ID NO: 16), and iTEP₁₁₂ (SEQ ID NO: 17) (Table 2). Next, the transition temperature (Tt) of each type of iTEP-IBD polypeptide was tested (FIGS. 1A and 1B). At the same concentration, the Tt of iTEP₅₆-IBD (SEQ ID NO: 36) was higher than iTEP₁₁₂-IBD (SEQ ID NO: 37) while lower than iTEP₂₈-IBD (SEQ ID NO: 38), which revealed a relation between MW and Tt of iTEP-IBD: the higher the MW, the lower the Tt. The results also showed that the Tt of each type of iTEP-IBD fusion polypeptide was a function of the concentration: the higher the concentration, the lower the Tt. In sum, the Tt of the iTEP-IBD polypeptide should be lower than 37° C. so that the iTEP-IBD polypeptide can transform to an insoluble phase and form depots after being inject into tissues. Next, it was examined whether the iTEP-IBD polypeptide can trap IgG at the depots. In this experiment, the mixture of the iTEP-IBD polypeptide and IgG were incubated at 37° C. to allow the formation of the depots. The depots were then collected to analyze the amount of contained IgG. It was found that the fraction of IgG in the depots was dependent on two factors: the MW of the iTEP-IBD polypeptide and the molar ratio of the iTEP-IBD polypeptide to IgG (FIG. 1C). When the ratio of iTEP₂₈-IBD to IgG was 8 or higher, about 55% of IgG was in depots. For iTEP₅₆-IBD and iTEP₁₁₂-IBD, when the ratio was 8 or higher, about 90% of IgG was in depots. These results suggest that the IgG in depots can be fine-tuned by controlling the ratio and the MW of the iTEP-IBD polypeptide. Since the iTEP-IBD polypeptide can bind to IgG, it was assessed whether the iTEP-IBD polypeptide could interfere with the target-binding ability of an antibody. For this study, the anti-PD-1 (αPD-1) antibody was used as the model antibody. EL4 cells, a cell line expressing PD-1 on the cell surface, was also used as the target cells. As shown by the flow cytometry results (FIGS. 1D and 1E), after binding with iTEP₁₁₂-IBD, the αPD-1 antibody can still bind to PD-1 on EL4 cells, similar to the free αPD-1 antibody. These results suggest that the iTEP-IBD polypeptide did not impact the target-binding ability of the antibodies.

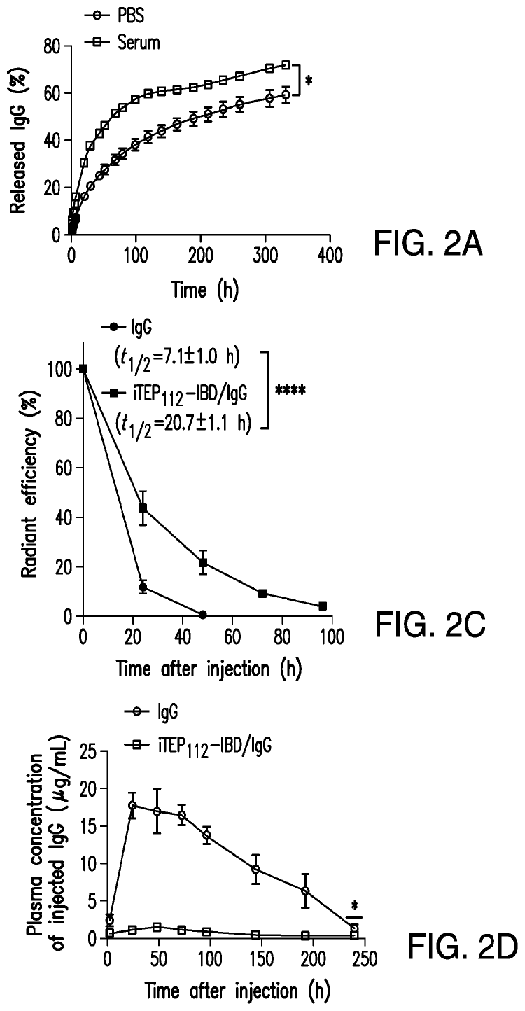

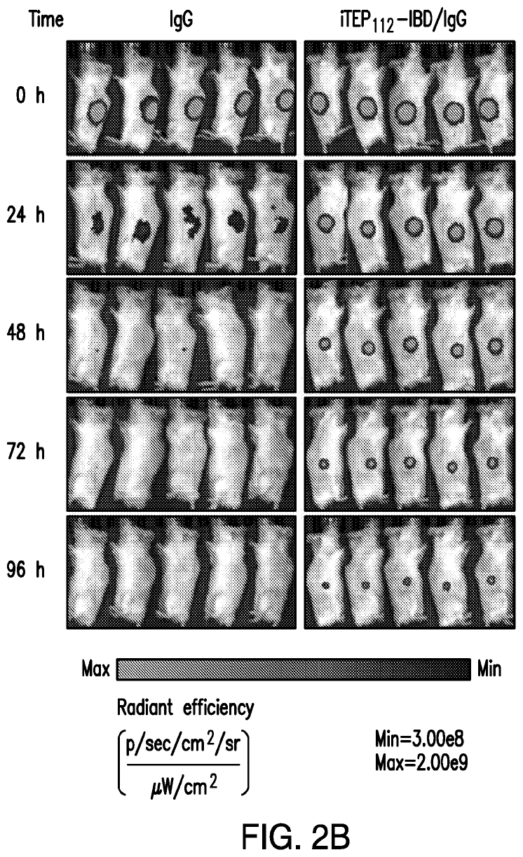

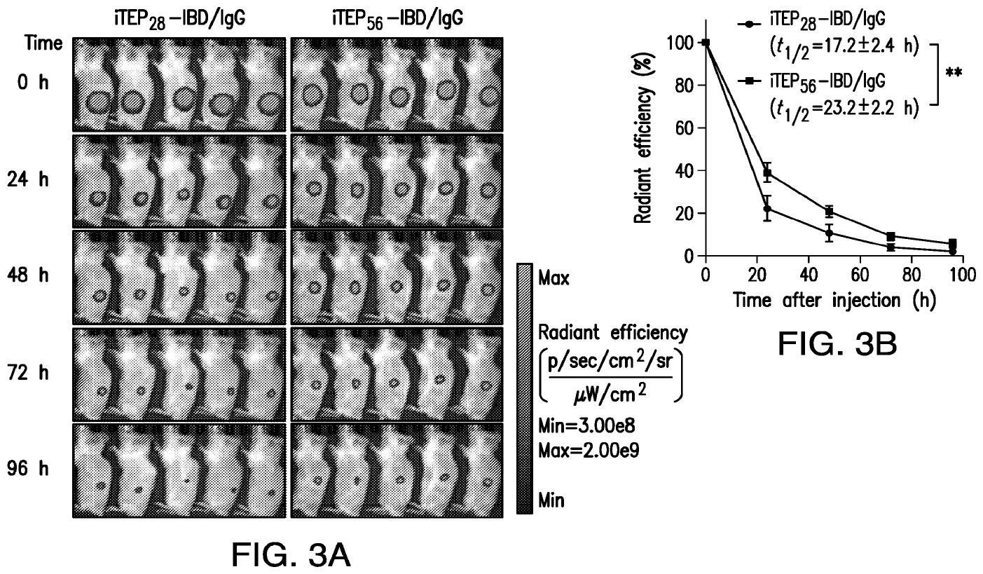

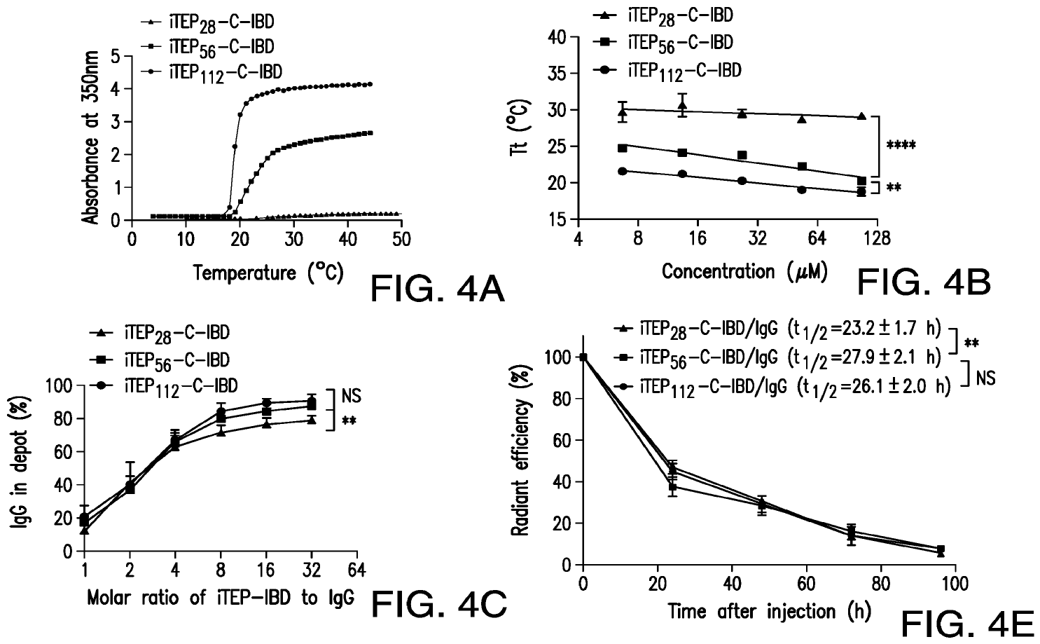

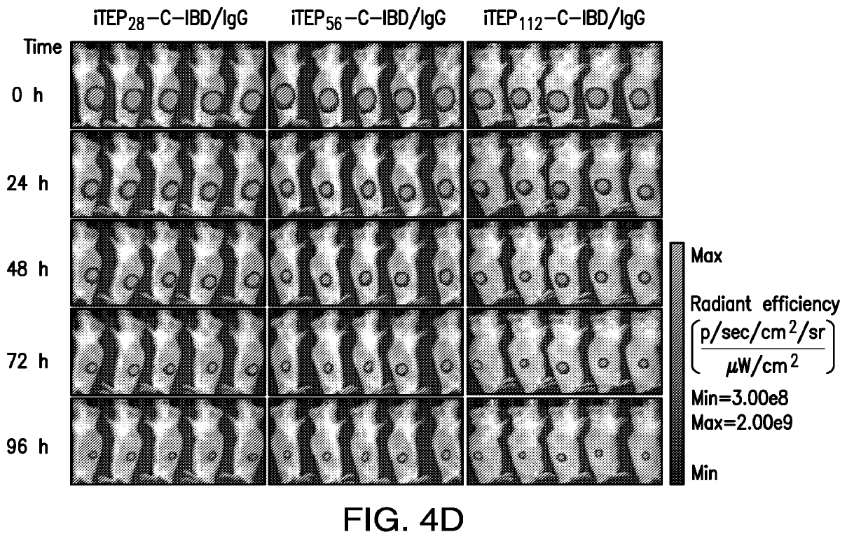

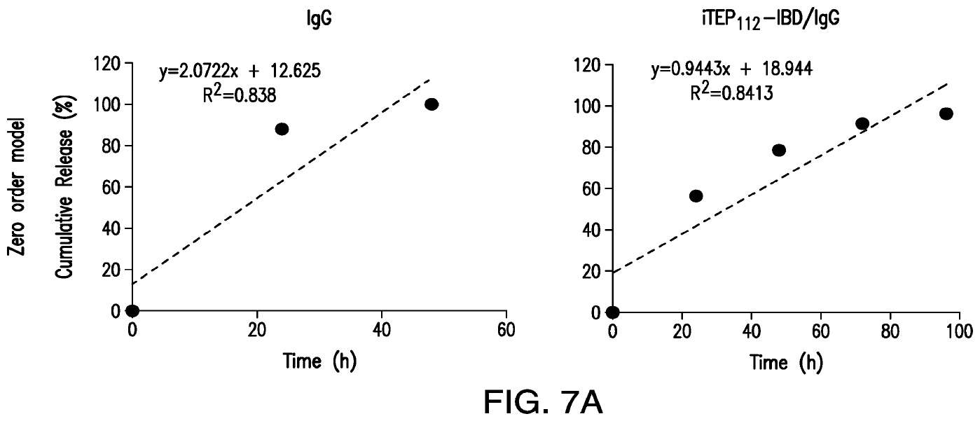

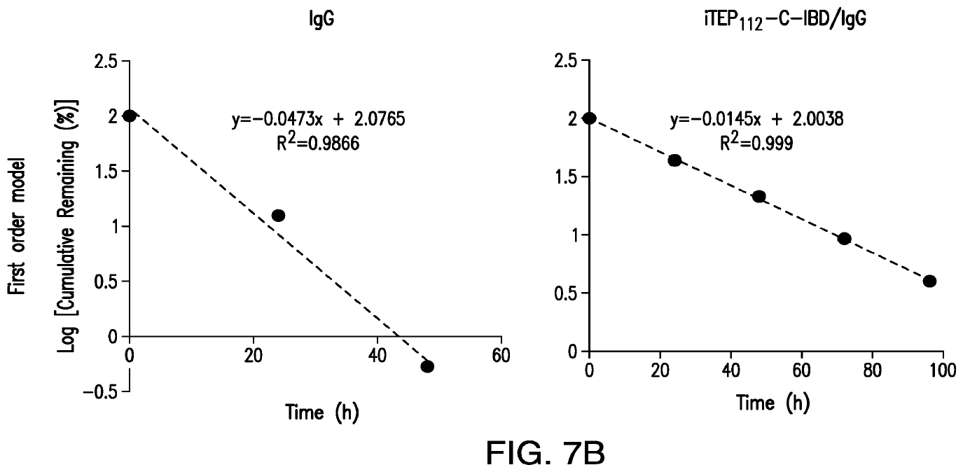

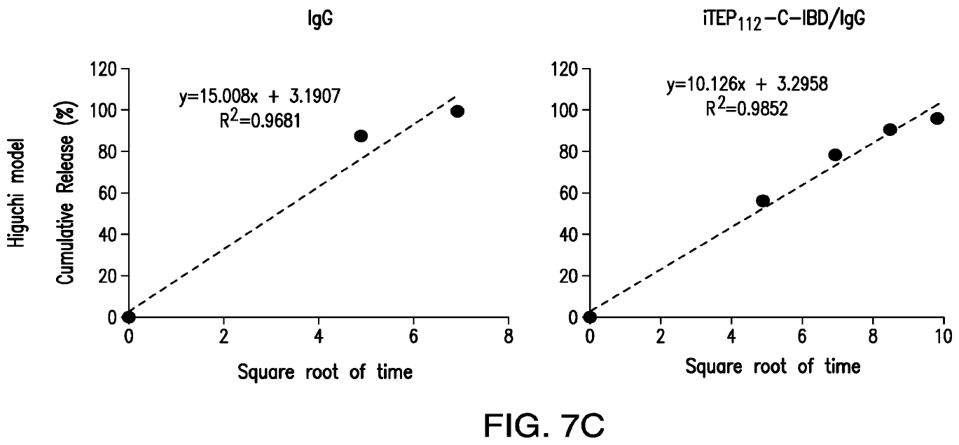

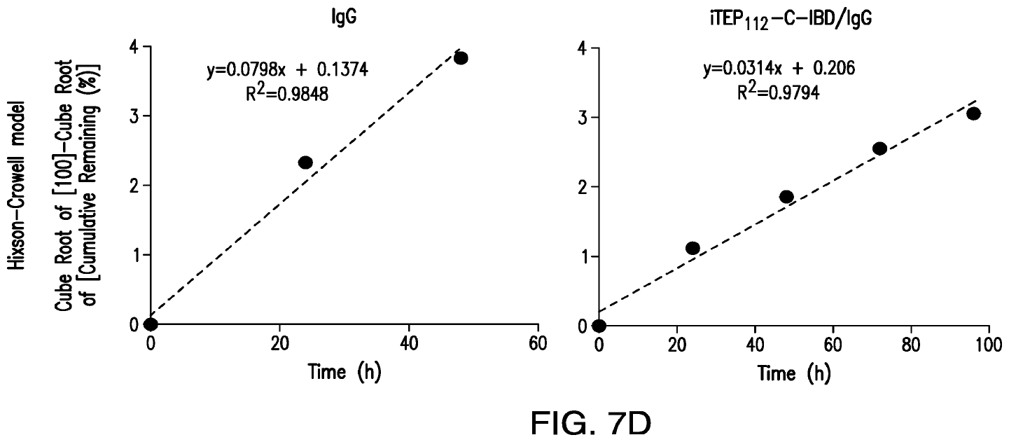

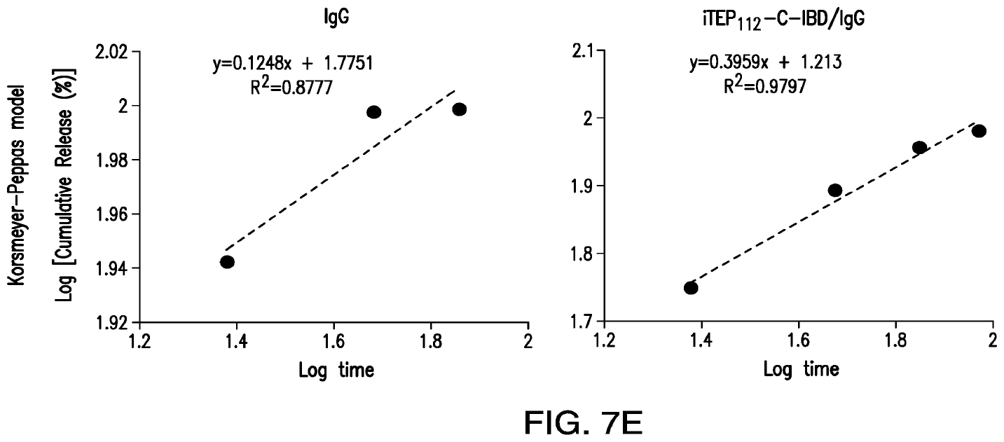

iTEP-IBD polypeptide sustained the release of IgG. Since the iTEP-IBD fusion polypeptide can trap IgG in depots, the release of IgG from the depots was then checked. The IgG release was first tested in vitro with two kinds of release buffer: PBS and 100% mouse serum. It was found that there was a burst release of IgG within the first 100 hours, followed by a steady release over a long time (FIG. 2A). The burst release may come from the bound IgG at the surface of the depots that were quickly immersed by the release buffer. The steady release may result from the bound IgG at the inside of depots since the release buffer took longer time to penetrate the depots. The burst release in mouse serum was more evident than in PBS, which was probably because proteases present in serum promoted the degradation of depots. The research found that proteases caused proteolytic degradation of proteins and peptides in serum and inhibitors of the proteases could reduce the degradation (J. Yi, et al., J Proteome Res 6(5) (2007) 1768-81; and R. Bottger, et al., PLoS One 12(6) (2017) e0178943). In addition, the mouse IgG in the serum may compete with the bound IgG for the binding to iTEP-IBD polypeptide and replace the bound IgG at the depots. This replacement may accelerate the IgG release and be another reason for the burst release in serum. Next IgG release at injection sites was examined in vivo. In this experiment, free IgG or the mixture of iTEP₁₁₂-IBD and IgG (iTEP₁₁₂-MD/IgG) were subcutaneously injected into mice and observed the remaining IgG at injection sites over time. The results show that iTEP₁₁₂-IBD keeps IgG at the injection sites for more than 96 hours compared to free IgG that disappeared from the injection sites after 24 hours (FIG. 2B). The fluorescent intensity of the remaining IgG at injection sites was quantified over time (FIG. 2C) and different mathematical models were used to analyze the release kinetics of IgG (FIG. 7). As indicated by the coefficient of determination of different models (Table 3), the first-order model was found to best describe the release profile of IgG and iTEP₁₁₂-MD/IgG in vivo. Therefore, the first-order model was used to analyze the IgG release in others experiments. Based on the analysis of the first-order model, the release half-life of IgG and iTEP₁₁₂-IBD/IgG was 7.1±1.0 h and 20.7±1.1 h, respectively (FIG. 2C). The plasma concentration of IgG after injection was also compared. When IgG was subcutaneously injected alone, the plasma concentration of IgG was much higher than that when IgG was injected together with iTEP₁₁₂-IBD (FIG. 4.2D). The area under the curve (AUC) of iTEP₁₁₂-1BD/IgG (106.9 μg/mL/h) was 13 times lower than the AUC of free IgG (1402.7 μg/mL/h). This data indicated that iTEP₁₁₂-IBD could decrease the systemic exposure of antibodies, which may reduce the risk of side effects of antibody treatment. Also, the release of iTEPs6-IBD/IgG and iTEP₂₈-IBD/IgG in vivo (FIG. 3A) was investigated. Based on the release kinetics (FIGS. 2C and 3B), iTEP₁₁₂-IBD/IgG and iTEP₅₆-IBD/IgG had similar release half-lives (20.7±1.1 h and 23.2±2.2 h, respectively), while iTEP₂₈-IBD/IgG had shorter release half-life (17.2±2.4 h), which was because iTEP₂₈-IBD had higher Tt than iTEP₁₁₂-IBD and iTEP₅₆-IBD (FIG. 1B).

TABLE 3
The coefficient of determination (R²) of different models that were used to
analyze the release kinetics of IgG and iTEP₁₁₂-IBD/IgG.

| | | | | Hixson- | Korsmeyer- |
|---|---|---|---|---|---|
| | Zero-order | First-order | Higuchi | Crowell | Peppas |
| IgG | 0.8380 | 0.9866 | 0.9681 | 0.9848 | 0.8777 |
| iTEP₁₁₂-IBD/IgG | 0.8413 | 0.9990 | 0.9852 | 0.9794 | 0.9797 |

Crosslinking of the iTEP-IBD fusion protein impacted the release of IgG. A previous study showed that the intermolecular crosslinking impacted the stability of iTEP (S. Dong, et al., Theranostics 6(5) (2016) 666-78). Therefore, it was tested whether the crosslinking of iTEP-IBD polypeptide may increase the stability of the depots, thus, impacting the release rate of IgG from depots. To crosslink the iTEP-IBD polypeptide, cysteine residues were introduced between the iTEP and the IBD (Table 1), and the new polypeptide was named iTEP-C-IBD. The cysteine residues were designed to form intermolecular disulfide bonds in oxidizing condition, thus crosslinking the iTEP-C-IBD polypeptide. After generating iTEP₂₈-C-IBD, iTEP₅₆-C-IBD, and iTEP₁₁₂-C-MD, their Tt (FIGS. 4A and 4B) was tested. It was observed that a drop of Tt after adding cysteine residues: the Tt of iTEP-C-IBD polypeptide was lower than the Tt of the corresponding iTEP-IBD polypeptide (FIGS. 1B and 4B). There was a 3-10° C. drop of Tt for iTEP₂₈-C-IBD in comparison with iTEP₂₈-IBD. The percentage of IgG in the iTEP-C-IBD depots was also examined. Comparing to iTEP₂₈-IBD, iTEP₂₈-C-IBD trapped a higher percentage of IgG in depots (FIGS. 1C and 4C). At the same time, iTEP₅₆-C-IBD and iTEP₁₁₂-C-IBD trapped a similar percentage of IgG in depots as iTEP₅₆-IBD and iTEP₁₁₂-IBD, respectively (FIGS. 1C and 4C). Then, the release of iTEP₂₈-C-IBD/IgG, iTEP₅₆-C-IBD/IgG, and iTEP₁₁₂-C-IBD/IgG was examined in vivo (FIG. 4D). iTEP₅₆-C-IBD/IgG had a similar release half-life with iTEP₁₁₂-C-IBD/IgG (27.9±2.1 h and 26.1±2.0 h, respectively) and a longer release half-life than iTEP₂₈-C-IBD/IgG (23.2±1.7 h) (FIG. 4E). Also, the release half-lives of iTEP-C-IBD/IgG mixture were longer than that of their corresponding iTEP-IBD/IgG mixture (FIGS. 2C, 3B, and 4E). These results demonstrated that crosslinking of the iTEP-IBD polypeptide could increase the release half-life of IgG.

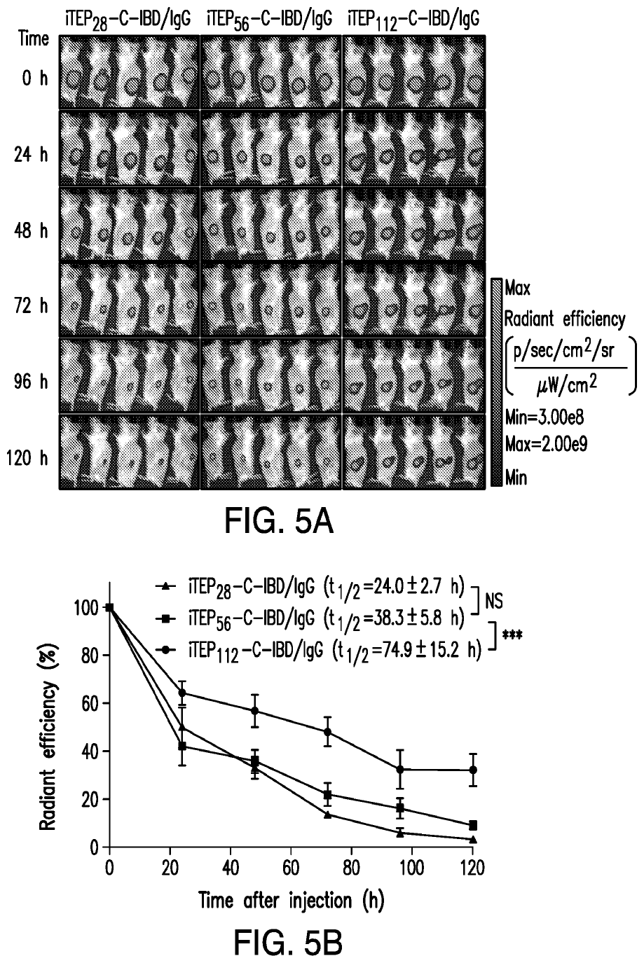

The ratio of the iTEP-C-IBD polypeptide to IgG impacted the release of IgG. In the release study of the iTEP-C-IBD/IgG mixture (and iTEP-MD/IgG mixture) as discussed herein, the ratio of iTEP-C-IBD polypeptide (and iTEP-IBD polypeptide) to IgG in the mixture was 8:1. It was then assessed whether the amount of the iTEP-C-IBD polyketide in the iTEP-C-IBD/IgG mixture could impact the IgG release. Therefore, the amount of IgG in the mixture was kept the same as previous studies while increasing the amount of the iTEP-C-IBD polypeptide to make the ratio of the iTEP-C-IBD polypeptide to IgG 32:1. The release was observed in vivo. The results show that iTEP₁₁₂-C-IBD/IgG had the longest release half-life (74.9±15.2 h), followed by iTEP₅₆-C-IBD/IgG (38.3±5.8 h) and iTEP₂₈-C-IBD/IgG (24.0±2.7 h) (FIGS. 5A and 5B). For the iTEP-C-IBD/IgG mixtures, the release half-lives increased at the ratio of 32:1 compared with the release half-lives at the ratio of 8:1 (FIGS. 4E and 5B). Moreover, the release half-life of iTEP₁₁₂-C-IBD/IgG mixture increased from 26.1±2.0 h to 74.9±15.2 h, with the change of the ratio from 8:1 to 32:1. These data demonstrate that the release half-life can be increased by increasing the ratio of the iTEP-C-IBD polypeptide to IgG. As the ratio increases, more iTEP-C-IBD polypeptides form depots, which may provide a shield to the IgGs present in the depots and slow down the release of the IgG.

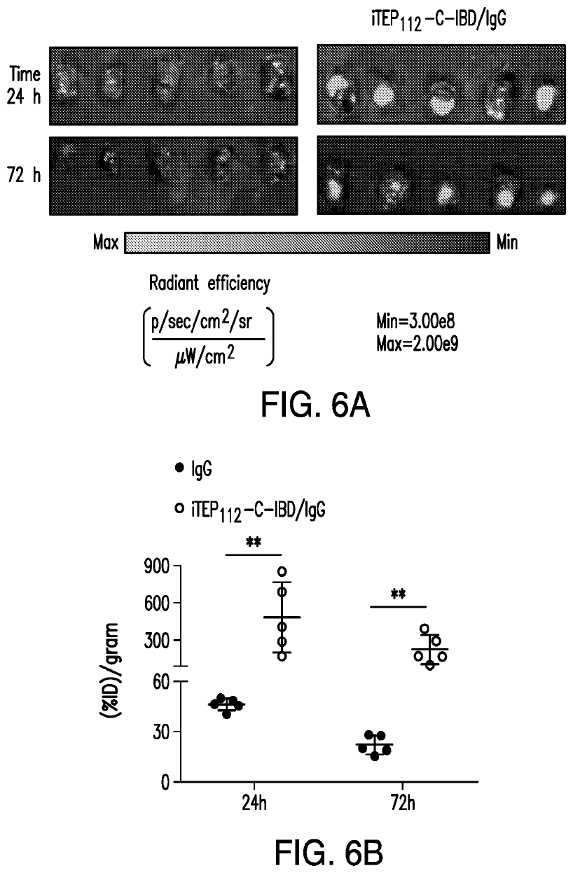

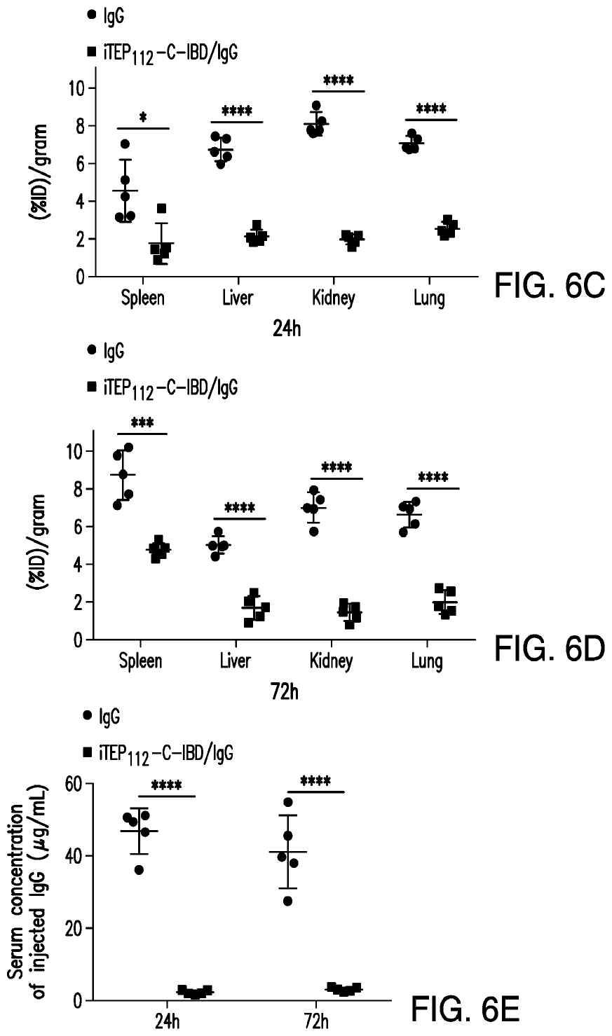

The iTEP-C-IBD polypeptide retained IgG in tumors and reduced systemic exposure. After studying the IgG release in vivo, it was tested whether the iTEP-C-IBD polypeptide can control the IgG release in a tumor model, e.g., melanoma. Previous research showed that intra-tumor injection of immune checkpoint antibodies, such as anti-PD-1 antibodies and anti-CTLA-4 antibodies, was effective in controlling tumor growth (A. Marabelle, et al., J Clin Invest 123(6) (2013) 2447-63; I. Sagiv-Barfi, et al., Sci Transl Med 10(426) (2018); and J. Ishihara, et al., Sci Transl Med 9(415) (2017)). But the free antibodies retained in the tumor for a short time and entered into systemic circulation quickly, which may render suboptimal effects and risk of side effects (F. Wu, et al., Pharm Res 29(7) (2012) 1843-53; and D. Schweizer, et al., Eur J Pharm Biopharm 88(2) (2014) 291-309). To solve these challenges, it was tested whether the iTEP-C-IBD polypeptide could keep the antibodies in the tumor and reduce their systemic exposure. Free IgG and iTEP₁₁₂-C-IBD/IgG mixture was injected into melanoma tumors and then observed the remaining IgG in tumors at different time points. First, IVIS imaging was used to visualize the remaining IgG in the tumor. It was found that there was more remaining IgG in the iTEP₁₁₂-C-IBD/IgG mixture group than that in the free IgG group at each time point (FIG. 6A). The remaining IgG in the tumor (FIG. 6B). At both time points, the remaining IgG in the iTEP₁₁₂-C-IBD/IgG mixture group was about 10 times more than that in the free IgG group, as indicated by the percentage of injected dose per gram tissue [(% ID)/gram]. Immune checkpoint antibodies can cause organ-specific toxicity because of the excessively activated immunity in normal organs (M. A. Postow, et al., N Engl J Med 378(2) (2018) 158-68; and J. M. Michot, et al., Eur J Cancer 54 (2016) 139-48). The antibodies can potentially cause toxicity in any organ, but the commonly affected organs include liver, kidney, lung, skin, endocrine glands, and hematologic systems. Some of these toxicities are fetal, such as pneumonitis, hepatitis, and myocarditis (F. Martins, et al., Nat Rev Clin Oncol (2019)). Limiting the exposure of immune checkpoint antibodies in these organs can reduce organ-specific toxicity. Therefore, the accumulation of IgG in organs, including spleen, liver, kidney, and lung and the blood, was examined. The results show that the amount of iTEP₁₁₂-C-lBD/IgG mixture was significantly less than that of free IgG in those organs (FIGS. 6C and 6D). Besides, the serum concentration of iTEP₁₁₂-C-IBD/IgG mixture was 20 and 13 times lower than that of free IgG at 24 and 72 hours after injection, respectively (FIG. 6E). These data revealed that iTEP₁₁₂-C-IBD polypeptide can keep antibodies in a tumor and limit the antibody exposure to other organs and systemic circulation.

Discussion. Local antibody treatments, such as immune checkpoint inhibitors, are drawing attention due to the advantages such as increased local bioavailability, reduced side effects, and inexpensive cost (R. G. Jones, A. Martino, Crit Rev Biotechnol 36(3) (2016) 506-20; K. Kitamura, et al., Cancer Res 52(22) (1992) 6323-8; A. D. Simmons, M. Moskalenko, J. Creson, J. Fang, S. Yi, M. J. VanRoey, J. P. Allison, K. Jooss, Local secretion of anti-CTLA-4 enhances the therapeutic efficacy of a cancer immunotherapy with reduced evidence of systemic autoimmunity, Cancer Immunol Immunother 57(8) (2008) 1263-70; D. W. Grainger, Expert Opin Biol Ther 4(7) (2004) 1029-44; and A. Marabelle, et al., Clin Cancer Res 19(19) (2013) 5261-3). However, the retention time of antibodies at local injection sites is short (F. Wu, et al., Pharm Res 29(7) (2012) 1843-53), which limits the therapeutic potential and requires frequent injections (D. Schweizer, et al., Eur J Pharm Biopharm 88(2) (2014) 291-309). Therefore, there is a need to develop an antibody delivery system that can retain antibody at injection for a longer time. As described herein, iTEP-IBD-based systems were developed that can form depots at body temperature after injection. Using the developed iTEP-IBD-based system, antibodies were trapped to depots through their binding with IBD. The depots could then control the antibody release over a long time.

A special feature of the iTEP-IBD-based systems is that the antibody release rate can be controlled. The results described herein show that three methods can be used to control the IgG release rate. First, the MW of the iTEP-IBD polypeptide can impact the Tt, thus regulating the IgG release rate. The iTEP₂₈-IBD/IgG mixture, the iTEP₅₆-IBD/IgG mixture, and the iTEP₁₁₂-IBD/IgG mixture were compared and the results show that the iTEP₅₆-IBD/IgG mixture and the iTEP₁₁₂-1BD/IgG mixture had similar IgG release half-lives, both of which were longer than that of the iTEP₂₈-IBD/IgG mixture. The shorter the IgG release half-life of the iTEP₂₈-IBD/IgG mixture was because of the higher Tt of the iTEP₂₈-IBD polypeptide. The iTEP₁₁₂-IBD polypeptide had a slightly lower Tt than the iTEP₅₆-IBD polypeptide, but they had similar IgG release half-lives, which was probably because the small difference in Tt did not result in a significant difference on the release half-life. Second, crosslinking of the iTEP-IBD polypeptide can impact the IgG release rate. The iTEP-C-IBD polypeptide was designed to contain cysteine residues so that the intermolecular disulfide bonds could cross-link the iTEP-C-IBD polypeptide. The results show that the IgG release half-life of the iTEP-C-IBD/IgG mixture was longer than that of the counterpart iTEP-IBD/IgG mixture. Intermolecular crosslinking may improve the stability of depots in vivo, thus increasing the release half-life. The third method to regulate IgG release was to control the ratio of the iTEP-C-IBD polypeptide to IgG. The IgG release half-life of the iTEP-C-IBD/IgG mixture at the ratio of 32:1 was longer than the half-life at the ratio of 8:1, which indicated that the IgG release half-life can be enhanced by increasing the ratio of the iTEP-C-IBD polypeptide to IgG. The increase of the ratio from 8:1 to 32:1 did not significantly increase the half-life of the iTEP₂₈-C-IBD/IgG mixture (23.2±1.7 h and 24.0±2.7 h, respectively). The reason for this result may be attributed to the Tt of the iTEP₂₈-C-IBD polypeptide. With the increase in the concentration of the iTEP-C-IBD polypeptide, the Tt of both the iTEP₅₆-C-IBD polypeptide and the iTEP₁₁₂-C-IBD polypeptide decreased, but the Tt of the iTEP₂₈-C-IBD polypeptide did not change (FIG. 4B). The concentration-independent Tt may explain why the increase of ratio did not significantly impact the release half-life of the iTEP₂₈-C-IBD/IgG mixture. At the same time, the release half-lives of the iTEP₅₆-C-IBD/IgG mixture and the iTEP₁₁₂-C-IBD/IgG mixture were similar at the ratio of 8:1 (27.9±2.1 h and 26.1±2.0 h, respectively), but quite different at the ratio of 32:1 (38.3±5.8 h and 74.9±15.2 h, respectively). The reason underlying this difference was not well-understood. The iTEP₁₁₂-C-IBD polypeptide had a lower Tt and was a longer length than the iTEP₅₆-C-IBD polypeptide. A possible explanation for the difference may be that the lower Tt and the longer length enhanced the half-life more significantly at the ratio of 32:1 than at the ratio of 8:1.

By combining these three methods, the IgG release half-life can be controlled from about 16 to about 64 hours. An antibody delivery system with tunable release rate is desirable. An acute ailment, such as infection, and a chronic symptom, such as rheumatoid arthritis, may need different release rates of antibodies. Even for the same type of disease, different stages of the disease may need a specific antibody release rate. These data suggest that the iTEP-IBD-based system represents an adjustable platform to meet different needs of different diseases and different disease states.

The results described herein demonstrate that the iTEP₁₁₂-C-IBD polypeptide can retain antibodies in a tumor for more than 72 hours. In this experiment, human IgG that did not have target-binding ability to tumor cells was used because the aim of the experiment was to examine how the iTEP₁₁₂-C-IBD polypeptide impacted the antibody retention in the tumor. If the antibodies can bind to membrane targets on tumor cells, their retention at the tumor may be more complicated. First, the binding of antibodies to the tumor targets can increase the accumulation and retention of the antibodies in tumor (C. F. Molthoff, et al., Br J Cancer 65(5) (1992) 677-83). In addition, if the antibody binding can trigger the target internalization, the bound antibodies can be internalized and degraded in cells (G. M. Thurber, et al., Trends Pharmacol Sci 29(2) (2008) 57-61). The clearance of these antibodies in tumor sites follows the pattern of target-mediated drug disposition, which is a non-linear pharmacokinetics profile (P. M. Glassman, J. P. Balthasar, Cancer Biol Med 11(1) (2014) 20-33). Their clearance is dependent on many factors, including the target density, internalization rate, turn over rate, and the binding affmities (W. Wang, et al., Clin Pharmacol Ther 84(5) (2008) 548-58). these factors can impact antibody retention if the antibodies can bind to tumor targets. By using antibodies without target-binding ability, these factors can be ruled out and the factor of the iTEP₁₁₂-C-IBD polypeptide on antibody retention was evaluated.

The data disclosed herein also provided evidence that the iTEP₁₁₂-C-IBD polypeptide reduced antibody exposure in the systemic circulation and other organs. Limiting antibody exposure to non-target organs is important to reduce side effects. Therapeutic antibodies, such as immune checkpoint inhibitors, are effective in treating melanoma (F. S. Hodi, et al., N Engl J Med 363(8) (2010) 711-23; C. Robert, et al., N Engl J Med 372(4) (2015) 320-30; and J. Dine, et al., Asia Pac J Oncol Nurs 4(2) (2017) 127-35). However, one challenge that limits the potential of immune checkpoint antibodies is immune-related side effects (R. M. Ruggeri, et al., J Endocrinol Invest (2018); J. Naidoo, et al., Ann Oncol 26(12) (2015) 2375-91; and I. Puzanov, et al., J Immunother Cancer 5(1) (2017) 95). The side effects were even more problematic when different immune checkpoint antibodies were combined for treatments. A clinical study showed that 55.0% of melanoma patients receiving the combination therapy of anti-PD-1 antibodies and anti-CTLA-4 antibodies had grade 3 or 4 side effects and 36.4% of the patients had to discontinue the therapy because of the side effects (J. Larkin, et al., N Engl J Med 373(1) (2015) 23-34). The side effects of immune checkpoint antibodies are organ-specific and cause toxicity in liver, lung, gastrointestinal tract, endocrine glands, etc. (A. Winer, et al., J Thorac Dis 10(Suppl 3) (2018) S480-9; and F. Martins, et al., Nat Rev Clin Oncol (2019)). The iTEP-IBD-based system described herein may reduce the organ-specific side effects by reducing the exposure of immune checkpoint antibodies in these organs. Besides, after reducing the side effects, higher doses of antibodies can be administered, which will, in turn, enhance the therapeutic efficacy.

The iTEP-IBD-based system is versatile because it can bind to a broad range of IgG subclasses through the IBD moiety (L. Bjorck, G. Kronvall, J Immunol 133(2) (1984) 969-74; and B. Akerstrom, et al., J Immunol 135(4) (1985) 2589-92). IBD is a 56-residue domain derived from protein G (B. Guss, et al., EMBO J 5(7) (1986) 1567-75; and A. M. Gronenborn, G. M. Clore, ImmunoMethods 2(1) (1993) 3-8). IBD can bind to both the fragment crystallizable (Fc) region and the fragment antigen-binding (Fab) region of IgG (M. Erntell, et al., Mol Immunol 25(2) (1988) 121-6). IBD binds to Fc at the hinge region between the CH2 and CH3 domains (A. E. Sauer-Eriksson, et al., Structure 3(3) (1995) 265-78; and K. Kato, et al., Structure 3(1) (1995) 79-85), and binds to Fab at the CH1 domain (M. Erntell, et al., Mol Immunol 25(2) (1988) 121-126; J. P. Derrick, D. B. Wigley, Nature 359(6397) (1992) 752-4; and J. P. Derrick, D. B. Wigley, J Mol Biol 243(5) (1994) 906-18). Its binding affinity for Fab is much weaker than its binding affinity for Fc (F. Unverdorben, et al., PLoS One 10(10) (2015) e013983). The antigen binding sites of an antibody are in the variable domains, while the IBD binding sites are in the constant domains, which may explain the observation that the iTEP-IBD polypeptide did not impair the antibody's binding ability to its target. Besides the variable domains, the Fc parts also mediate effector functions of antibodies, such as complement-dependent cytotoxicity, antibody-dependent cellular cytotoxicity, and antibody-dependent cellular phagocytosis (C. Kellner, et al., Transfus Med Hemother 44(5) (2017) 327-36; X. Wang, et al., Protein Cell 9(1) (2018) 63-73; and S. Bournazos, Cell 158(6) (2014) 1243-53). It is not known whether the iTEP-IBD may impact the Fc-mediated functions of an antibody. For some antibodies, such as αPD-1 antibody (R. Dahan, et al., Cancer Cell 28(3) (2015) 285-95; and T. Zhang, et al., Cancer Immunol Immunother 67(7) (2018) 1079-90), their effector mechanisms are not dependent on the Fc. Therefore, the iTEP-IBD-based system can be at least applied to deliver those antibodies without diminishing their function.

In sum, a versatile system for local delivery of antibodies was developed. This system can be used to increase the therapeutic effects and reduce the side effects of antibodies.

Materials and Methods. Animals and cell lines. Six-week-old female BALB/c mice weighing 19.1±1.2 g and six-week-old female C57BL/6 mice weighing 17.5±1.0 g were purchased from the Jackson Laboratory. EL4 cells (American Type Culture Collection) were cultured with DMEM medium supplemented with 10% horse serum. B16-F10 cells (American Type Culture Collection) were cultured in DMEM medium supplemented with 10% fetal bovine serum, 100 μg/mL streptomycin, and 100 U/mL penicillin. Cells were cultured at 37° C. with 95% air and 5% carbon dioxide.

Expression of iTEP-based polypeptides. The DNA sequences coding for iTEP and IBD were synthesized (Eurofins Genomics) and inserted into plasmids using the cloning method (P. Wang, et al., Theranostics 8(1) (2018) 223-36; and S. Cho, et al., J Drug Target 24(4) (2016) 328-39)). The plasmids were then transferred to BL21 (DE3) competent E. coli cells for the expression of polypeptides. The polypeptides were purified (S. Dong, et al., Acta Pharmacol Sin 38(6) (2017) 914-23; and S. Dong, et al., Mol Pharm 14(10) (2017) 3312-21). The endotoxin level in the polypeptides was under 0.25 EU/mg for in vivo study (P. Wang, et al., Biomaterials 182 (2018) 92-103).

Characterization of the Tt of the polypeptides. The optical density at 350 nm (OD350) of each polypeptide solution at different concentrations was monitored over a temperature range from 4-50° C. using a UV—visible spectrophotometer (Varian Instruments). Sigmoidal dose-response nonlinear regression (GraphPad, version 6.01) was used to fit the curve between the OD350 and the temperature. The maximum first derivative of the curve was determined as the Tt.

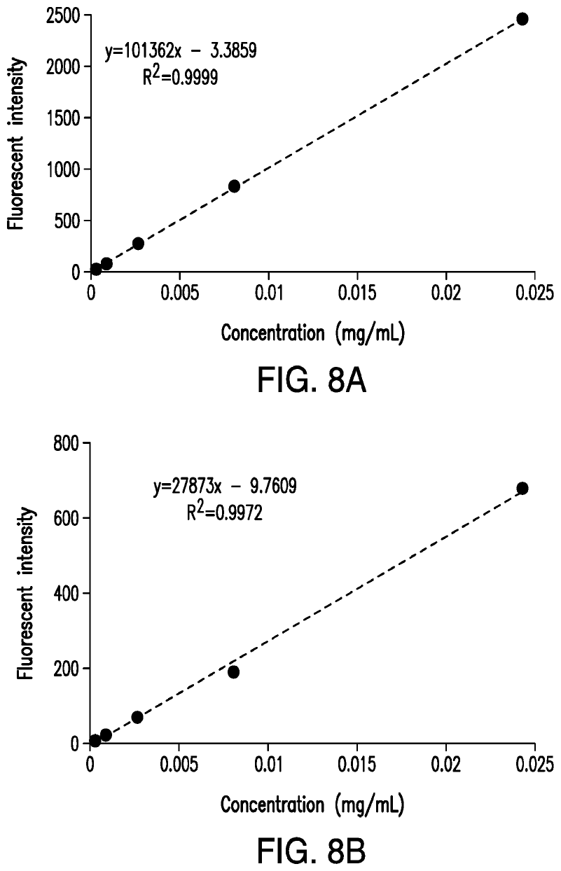

Determining the percentage of IgG trapped by the iTEP-IBD polypeptide. Human IgG with purity greater than 97% was purchased from Innovative Research. The human IgG was polyclonal and contained subclasses IgG1, IgG2, IgG3, and IgG4. The human IgG was purified from human plasma or serum by fractionation. The human IgG was labeled with NHS-Fluorescein (Thermo Fisher Scientific). The labeled IgG and free fluorescein were separated by PD-10 desalting columns with Sephadex G-25 resin (GE Healthcare) for two times. The labeled IgG was concentrated through ultrafiltration centrifugation with Vivaspin spin columns (Molecular mass cut-off: 10,000 kDa, GE Healthcare). A standard curve depicting the linear correlation between the fluorescent intensity and the concentration of the fluorescein-labeled IgG solution in PBS was established (FIG. 8A). The fluorescent signal of the lowest IgG concentration in the standard curve was 20-fold higher than the background signal. iTEP, the iTEP-IBD polypeptide, and the iTEP-C-IBD polypeptide were incubated with the labeled IgG (1 mg/mL) at the designated ratios at 4° C. for overnight. Next, the mixture was incubated at 37° C. for 10 minutes and then centrifuged at 20,000 g for 10 minutes. After centrifugation, the pellets were collected and dissolved in PBS solution. The solution was transferred to a 96-well plate to examine the fluorescent intensity (excitation 494 nm, emission 518 nm) using the Infinite M1000 pro microplate reader (Tecan). The fluorescent intensity was converted to the IgG concentration based on the standard curve.

Antibody binding function assay. EL4 cells express PD-1 on the cell surface and can be stained by the αPD-1 antibody. The iTEP₁₁₂-IBD polypeptide was incubated with PE anti-mouse αPD-1 antibody (BioLegend, clone: RMP1-14) at a ratio of 2000:1 at 4° C. overnight. The iTEP₁₁₂-IBD/αPD-1 mixture and the free αPD-1 antibody were then used to stain EL4 cells. Previously it was shown that the isotype control antibody did not stain the EL4 cells, similar to the no staining control (P. Zhao, et al., Nat Biomed Eng 3(4) (2019) 292-305). Therefore, the isotype control antibody was not included in this experiment. The cells were then counted and analyzed by flow cytometry. The percentage of the stained EL4 cells indicated the target binding ability of the iTEP₁₁₂-IBD/αPD-1 mixture and free αPD-1 antibody.

Examining the IgG release in vitro. The iTEP₁₁₂-IBD polypeptide and the fluorescein-labeled IgG (1 mg/mL) at the ratio of 8:1 and a total volume of 100 μL were incubated at 4° C. overnight. The iTEP₁₁₂-IBD/IgG mixture was then incubated at 37° C. and centrifuged to collect the pellets. Next, the pellets were added to 100 μL PBS or 100% mouse serum. The mouse serum was prepared from the mouse blood without heat-inactivation, keeping the intact complement system and other serum components. The IgG-antigen immune complex may stimulate the classical pathway of the complement system (M. Noris, G. Remuzzi, Semin Nephrol 33(6) (2013) 479-92). But since the human IgG used in this experiment had no antigen-binding ability and could not form the IgG-antigen complex, the complement system in the mouse serum would not be activated or impact the IgG release. At each time point, the PBS or mouse serum was taken out to measure the fluorescent intensity to quantify the released IgG. Meanwhile, the pellets were added with 100 μL new PBS or mouse serum. The fluorescent background of mouse serum was subtracted before the fluorescent intensity was used to quantify the released IgG in mouse serum using the standard curve as described herein.

Examining the IgG release in vivo. Human IgG was labeled with sulfo-cyanine7 NHS ester (Lumiprobe). The free dye was removed by PD-10 desalting columns, and the labeled IgG was concentrated with Vivaspin spin columns as described herein. The iTEP-IBD polypeptide or the iTEP-C-IBD polypeptide was incubated with the sulfo-cyanine7-labeled IgG at 4° C. overnight. The iTEP-C-IBD/IgG mixture was then oxidized with 0.3% H₂O₂ overnight. BALB/c mice were shaved and subcutaneously injected with 100 μL free IgG (lmg/mL), the iTEP-IBD/IgG mixture (equivalent amount of IgG), or the iTEP-C-IBD/IgG mixture (equivalent amount of IgG) at the flank. The IgG used in this study was labeled with sulfo-cyanine7, a near-infrared dye with minimal autofluorescence, to reduce the tissue background (E. A. Owens, et al., Acc Chem Res 49(9) (2016) 1731-40; and P. S. Chan, et al., AAPS J 21(4) (2019) 59). The mice were imaged (excitation 745 nm, emission 800 nm, exposure 1 s) by IVIS Spectrum (Caliper Life Sciences) every 24 hours starting immediately after the injection. The radiant efficiency of injection sites was quantified by IVIS analysis software. The scale of fluorescence was adjusted to omit the influence of tissue autofluorescence before quantifying the radiant efficiency of injection sites. The radiant efficiency over the time was used to describe the release kinetics of IgG in vivo.

Detecting the plasma concentration of the injected IgG. A standard curve between the fluorescent intensity and the concentration of sulfo-cyanine7-labeled IgG was made (FIG. 8B). The fluorescent signal of the lowest IgG concentration in the standard curve was 6-fold higher than the background signal. C57BL/6 mice were subcutaneously injected with 100 μL sulfo-cyanine7-labeled IgG (1 mg/mL) or the iTEP₁₁₂-IBD/IgG mixture (equivalent amount of IgG) at the flank. At each time point, three drops of blood from each mouse were collected to a tube that was coated with ethylenediaminetetraacetic acid (EDTA). The tubes were then centrifuged at 20,000 g for 10 minutes to collect the plasma. The plasma was diluted in PBS to examine the fluorescent intensity (excitation 750nm, emission 773 nm) using the Infinite M1000 pro microplate reader (Tecan). The fluorescent background of the plasma was subtracted before the fluorescent intensity was converted to the IgG concentration through the standard curve.

Determining the amount of IgG retention in tumors and accumulation in other organs. C57BL/6 mice were intradermally injected with 5×10⁵ B16-F10 cells in 50 μL PBS at the flank. When the tumor diameter was about 0.5 cm, 50 μL sulfo-cyanine7-labeled IgG (2 mg/mL), or the iTEP₁₁₂-C-IBD/IgG mixture (equivalent amount of IgG) was directly injected into the tumor. At 24 and 72 hours after the injection, mice were euthanized. Tumors and other organs, including spleen, liver, kidney, and lung were collected. The tumors were imaged (excitation 745 nm, emission 800 nm, exposure 1 s) by IVIS Spectrum. The collected tumors and organs were weighed and homogenized in PBS. The homogenate was centrifuged to gather the supernatant and to measure the fluorescent intensity. The fluorescent background of the organs was subtracted from the fluorescent intensity, and the amount of IgG in the supernatant was quantified by referencing the standard curve as described herein. Blood was also collected from mice just before euthanasia. The blood was kept at room temperature for 30 minutes and then centrifuged to obtain serum. The serum was diluted in PBS to examine the fluorescent intensity. The fluorescent background of serum was subtracted from the fluorescent intensity, and the serum concentration of injected IgG was quantified by referencing the standard curve as described herein.

Statistics. Detailed statistics of each experiment is described in each figure legend. Unpaired two-tailed Student's t-test and one-way ANOVA test were used to analyze the data. P<0.05 was defined as a significant difference.

1. A recombinant polypeptide comprising an homologous amino acid repeat sequence, having at least 75% amino acid sequence identity to the homologous amino acid repeat sequence, and wherein the homologous amino acid repeat sequence is:
(SEQ ID NO: 1)
Gly-Val-Leu-Pro-Gly-Val-Gly;
(SEQ ID NO: 2)
Gly-Ala-Gly-Val-Pro-Gly;
(SEQ ID NO: 3)
Val-Pro-Gly-Phe-Gly-Ala-Gly-Ala-Gly;
(SEQ ID NO: 4)
Val-Pro-Gly-Leu-Gly-Ala-Gly-Ala-Gly;
(SEQ ID NO: 5)
Val-Pro-Gly-Leu-Gly-Val-Gly-Ala-Gly;
(SEQ ID NO: 6)
Gly-Val-Leu-Pro-Gly-Val-Gly-Gly;
(SEQ ID NO: 7)
Gly-Val-Leu-Pro-Gly;
(SEQ ID NO: 8)
Gly-Leu-Val-Pro-Gly-Gly;
(SEQ ID NO: 9)
Gly-Leu-Val-Pro-Gly;
(SEQ ID NO: 10)
Gly-Val-Pro-Leu-Gly;
(SEQ ID NO: 11)
Gly-Ile-Pro-Gly-Val-Gly;
(SEQ ID NO: 12)
Gly-Gly-Val-Leu-Pro-Gly;
or
(SEQ ID NO: 14)
Gly-Val-Gly-Val-Leu-Pro-Gly;
(SEQ ID NO: 15)
Gly-Val-Pro-Gly;
and
an IgG binding domain.

2. (canceled)

3. The recombinant polypeptide of claim 1, wherein the homologous amino acid repeat sequence is repeated linearly.

4. (canceled)

5. The recombinant polypeptide of claim 1, wherein the homologous amino acid repeat sequence is (Gly-Val-Leu-Pro-Gly-Val-Gly)28 (SEQ ID NO: 13); (Gly-Val-Leu-Pro-Gly-Val-Gly)₅₆ (SEQ ID NO: 16); or (Gly-Val-Leu-Pro-Gly-Val-Gly)₁₁₂ (SEQ ID NO: 17).

6. (canceled)

7. The recombinant polypeptide of claim 1, wherein the IgG binding domain comprises the sequence or is at least 75% identical to the amino acid sequence
(SEQ ID NO: 18)
TTYKLVINGKTLKGETTTKAVDAETAEK
AFKQYANDNGVDGVWTYDDATKTFTVTE.

8. The recombinant polypeptide of claim 1, further comprising one or more linker sequences.

9. (canceled)

10. (canceled)

11. The recombinant polypeptide of claim 8, wherein the recombinant polypeptide comprises the amino acid sequence (GVLPGVG)₂₈-GGGGS-TTYKLVINGKTLKGETTTKAVDAETAEKAFKQYANDNGVDGVWTYDDATK TFTVTE (SEQ ID NO: 36); (GVLPGVG)₅₆-GGGGS-TTYKLVINGKTLKGETTTKAVDAETAEKAFKQYANDNGVDGVWTYDDATK TFTVTE (SEQ ID NO: 37); (GVLPGVG)₁₁₂-GGGGS-TTYKLVINGKTLKGET TTKAVDAETAEKAFKQYANDNGVDGVWTYDDATKTFTVTE (SEQ ID NO:

38. ; (GVLPGVG)₂₈-(GGGGC)₄-GGGGS-TTYKLVINGKTLKGETTTKAVDAETAEKAFKQYANDNGVDGVWTYDDATK TFTVTE (SEQ ID NO: 41); (GVLPGVG)₅₆-(GGGGC)₄-GGGGS-TTYKLVINGKTLKGETTTKAVDAETAEKAFKQYANDNGVDGVWTYDDATK TFTVTE (SEQ ID NO: 42); or (GVLPGVG)₁₁₂-(GGGGC)₄-GGGGS-TTYKLVINGKTLKGETTTKAVDAETAEKAFKQYANDNGVDGVWTYDDATK TFTVTE (SEQ ID NO: 43).

12.-18. (canceled)

19. The recombinant polypeptide of claim 1, further comprising and one or more therapeutic agents, wherein the therapeutic agent is an anti-PD-1 antibody, anti-PD-L1 antibody, or an anti-CTLA-4 antibody.

20. The recombinant polypeptide of claim 19, wherein the one or more therapeutic agents are non-covalently bound to the IgG binding domain.

21.-26. (canceled)

27. A pharmaceutical composition comprising the recombinant polypeptide of claim 1 and a pharmaceutically acceptable carrier.

28. (canceled)

29. A method of treating a subject with cancer, the method comprising: administering to the subject a therapeutically effective amount of the pharmaceutical composition of claim 27, wherein the pharmaceutical composition further comprises one or more therapeutic agents.

30.-35. (canceled)

36. The method of claim 19, wherein the therapeutic agent has increased half-life, increased efficacy or reduced side effects when administered non-covalently bound to the recombinant polypeptide as compared to when the therapeutic agent is administered alone or not bound to the recombinant polypeptide.

37. (canceled)

38. A method of reducing tumor size in a subject in need thereof, the method comprising administering to the subject an effective amount of a composition comprising: the recombinant polypeptide of claim 1, wherein the IgG binding domain is non-covalently bound to a therapeutic agent, thereby reducing tumor size.

39. (canceled)

40. The method of claim 38, wherein the tumor is a malignant tumor, and the malignant tumor is breast cancer, ovarian cancer, lung cancer, colon cancer, gastric cancer, head and neck cancer, glioblastoma, renal cancer, cervical cancer, peritoneal cancer, kidney cancer, pancreatic cancer, brain cancer, spleen cancer, prostate cancer, urothelial carcinoma, skin cancer, myeloma, lymphoma, or a leukemia.

41.-50. (canceled)

51. The method of claim 38, wherein the therapeutic agent is an anti-PD-1 antibody, anti-PD-L1 antibody, or an anti-CTLA-4 antibody.

52.-57. (canceled)

58. A method of increasing the efficacy of a therapeutic agent or increasing the half of a therapeutic agent in a subject, the method comprising administering to the subject a therapeutic agent conjugated to the recombinant polypeptide of claim 1 comprises the homologous amino acid repeat sequence is covalently linked to a IgG binding domain, and wherein the therapeutic agent is non-covalently conjugated to the IgG binding domain, and wherein the conjugate is administered by direct injection, whereby the efficacy or half-life of the therapeutic agent is increased.

59. The method of claim 58, wherein the therapeutic agent is an anti-PD-1 antibody, anti-PD-L1 antibody, or an anti-CTLA-4 antibody.

60.-66. (canceled)

67. The method of claim 58, wherein the subject has cancer.

68. (canceled)

69. The method of claim 67, wherein the cancer is lung cancer, colon cancer, breast cancer, brain cancer, liver cancer, prostate cancer, spleen cancer, muscle cancer, ovarian cancer, pancreatic cancer, skin cancer, or melanoma.

70.-79. (canceled)
